(19) United States
(12) Patent Application Publication
(10) Pub. No.: US 2009/0022771 A1
(43) Pub. Date: Jan. 22, 2009

(54) BIOMATERIAL

(75) Inventors: Andrew K. Lynn, Cambridge (GB); William Bonfield, Cambridge (GB); Lorna J. Gibson, Cambridge, MA (US); Ioannis Yannas, Cambridge, MA (US); Brendan A. Harley, Cambridge, MA (US)

Correspondence Address: SENNIGER POWERS LLP, 100 NORTH BROADWAY, 17TH FLOOR, ST LOUIS, MO, 63102, US

(73) Assignees: CAMBRIDGE ENTERPRISE LIMITED, Cambridge (GB); MASSACHUSETTS INSTITUTE OF TECHNOLOGY, Cambridge, MA (US)

(21) Appl. No.: 11/908,045

(22) PCT Filed: Mar. 6, 2006
(86) PCT No.: PCT/GB2006/000797

§ 371 Date: Apr. 25, 2008

(30) Foreign Application Priority Data
Mar. 7, 2005 (GB) 0504673.5

(51) Int. Cl. A61F 2/28; A61K 31/726; A61P 19/00; B29C 67/20

(52) U.S. Cl. 424/423; 514/54; 264/42

(57) ABSTRACT

A process for the preparation of a composite biomaterial comprising an inorganic material and an organic material, the process comprising: (a) providing a first slurry composition comprising a liquid carrier, an inorganic material and an organic material; (b) providing a mould for the slurry; (c) depositing the slurry in the mould; (d) cooling the slurry deposited in the mould to a temperature at which the liquid carrier transforms into a plurality of solid crystals or particles; (e) removing at least some of the plurality of solid crystals or particles by sublimation and/or evaporation to leave a porous composite material comprising an inorganic material and an organic material; and (f) removing the material from the mould.

The present invention relates to the field of synthetic bone materials for biomedical applications and, in particular, to porous monolithic and porous layered scaffolds comprising collagen, calcium phosphate, and optionally a glycosaminoglycan for use in tissue engineering.

Natural bone is a biocomposite of collagen, non-collagenous organic phases including glycosaminoglycans, and calcium phosphate. Its complex hierarchical structure leads to exceptional mechanical properties including high stiffness, strength, and fracture toughness, which in turn enable bones to withstand the physiological stresses to which they are subjected on a daily basis. The challenge faced by researchers in the field is to make a synthetic material that has a composition and structure that will allow natural bone growth in and around the synthetic material in the human or animal body.

It has been observed that bone will bond directly to calcium phosphates in the human body (a property referred to as bioactivity) through a bone-like apatite layer formed in the body environment. Collagen and copolymers comprising collagen and other bioorganics such as glycosaminoglycans on the other hand, are known to be optimal substrates for the attachment and proliferation of numerous cell types, including those responsible for the production and maintenance of bone in the human body.

Hydroxyapatite is the calcium phosphate most commonly used as a constituent in bone substitute materials. It is, however, a relatively insoluble material when compared to other forms of calcium phosphate materials such as brushite, tricalcium phosphate and octacalcium phosphate. The relatively low solubility of apatite can be a disadvantage when producing a biomaterial as the rate of resorption of the material in the body is particularly slow.

Calcium phosphates such as hydroxyapatite are mechanically stiff materials. However, they are relatively brittle when compared to natural bone. Collagen is a mechanically tough material, but has relatively low stiffness when compared to natural bone. Materials comprising copolymers of collagen and glycosaminoglycans are both tougher and stiffer than collagen alone, but still have relatively low stiffness when compared to natural bone.

Previous attempts to produce a synthetic bone-substitute material having improved mechanical toughness over hydroxyapatite and improved stiffness over collagen and copolymers of collagen and glycosaminoglycans include combining collagen and apatite by mechanical mixing. Such a mechanical method is described in EP-A-0164 484.

Later developments include producing a bone-replacement material comprising hydroxyapatite, collagen and chondroitin-4-sulphate by the mechanical mixing of these components. This is described in EP-A-0214070. This document further describes dehydrothermic crosslinking of the chondroitin-4-sulphate to the collagen. Materials comprising apatite, collagen and chondroitin-4-sulphate have been found to have good biocompatibility. The mechanical mixing of the apatite with the collagen, and optionally chondroitin-4-sulphate, essentially forms collagen/chondroitin-4-sulphate-coated particles of apatite. It has been found that such a material, although biocompatible, produces limited in-growth of natural bone when in the human or animal body and no remodeling of the calcium phosphate phase of the synthetic material.

The repair of skeletal sites compromised by trauma, deformity or disease poses a special challenge to orthopaedic surgeons in that, unlike defects in skin, nerve and most other tissue types, skeletal defects encompass multiple, distinct tissue types (i.e. bone, cartilage, tendon and ligament), involve locations that undergo regular mechanical loading, and traverse interfaces between mineralised to unmineralised tissues (e.g. ligament insertion points, the “tidemark” at the bone/cartilage interface).

Existing clinical approaches address the repair of skeletal defects either with non-resorbable prosthetic implants, autologous or allogenous tissue grafts, chemical agents, cell transplantation or combinations of these methods. While these approaches have achieved some success for the treatment of single tissue types, cases where interfaces between mineralised and unmineralised tissue are involved, such as articular joint defects for example, result in healing that is, at best, incomplete. Furthermore, even the most successful of the existing treatments require either the harvest of tissue from a donor site and/or the suturing to bone, cartilage, ligament or tendon. The former procedure suffers from lack of donor sites and donor site morbidity, while the latter is difficult to implement and creates additional defects in the form of suture holes.

The terms composite scaffold and layered scaffold are synonymous, and refer to scaffolds comprising two or more layers, with the material composition of each layer differing substantially from the material composition of its adjacent layer or layers. The term single-layered scaffold or monolithic scaffold are synonymous, and refer to scaffolds comprising one layer only, with the material composition within each layer being largely homogeneous throughout.

A limited number of recent efforts have sought to develop tissue-engineering strategies that employ porous, layered scaffolds for the treatment of articular joint defects involving either cartilage alone or both bone and cartilage. These constructs seek to induce the regeneration of bone and cartilage concurrently, but using separate scaffolds for each (Niederauer et al., 2000; Schaefer et al., 2000; Gao et al., 2001; Gao et al., 2002; Schaefer et al., 2002; Sherwood et al., 2002; Hung et al., 2003; Hunziker and Driesang, 2003).

An additional feature of layered scaffolds is the potential they hold for achieving sutureless fixation via direct attachment of the bony layer to the subchondral bone plate. Provided the cartilaginous portion remains firmly attached to the bony portion, no additional fixation is required. Sutureless fixation may also enable the treatment of defects involving insertions points of tendon and ligament to bone. Despite the promise of this new approach, two shortcomings can limit the effectiveness of the layered scaffolds reported to date. The first relates to the materials used for the respective layers of the scaffold. Resorbable synthetic polymers have been the only material used for the cartilaginous layer, and have often been a component of the osseous portion in many of these scaffolds as well. Although easy to fabricate, synthetic polymers are known to be less conducive to cell attachment and proliferation than natural polymers such as collagen, and can furthermore release high concentrations of acid as they degrade. Moreover, for applications where tendon or ligament repair is necessary, resorbable synthetic polymers, regardless of the manner in which they are crosslinked, have inadequate strength and stiffness to withstand even the reduced load applied during rehabilitation exercises.

The second shortcoming of conventional layered scaffolds relates to the interface between the respective layers. Natural articular joints and tendon/ligament insertion points are characterised by continuity of collagen fibrils between the mineralised and unmineralised regions. The resultant system of smooth transitions (soft interfaces) imparts an intrinsic mechanical stability to these sites, allowing them to withstand physiological loading without mechanical failure. In contrast, the majority of existing layered scaffolds contain hard interfaces, forming a distinct boundary between two dissimilar materials. Suturing (Schaefer et al., 2000), fibrin adhesive bonding (Gao et al., 2001) and other techniques (Gao et al., 2002; Hung et al., 2003) have been used to strengthen this interface. However, interfacial debonding has still been reported even in controlled animal models. These suturing and bonding methods are also delicate and poorly reproducible.

Previous work has developed means through which the parameters of freeze-drying protocols can be controlled to produce porous scaffolds of collagen and one or more glycosaminoglycans (GAGs) (Yannas et al., 1989; O'Brien et al., 2004; O'Brien et al., 2005; Loree et al 1989).). These techniques allow scaffold features such as pore size and aspect ratio to be varied in a controlled manner, parameters known to have marked effects on the healing response at sites of trauma or injury. However, for treatment of injuries involving skeletal and musculoskeletal defects, it is necessary to develop technologies to produce porous scaffolds with material compositions and mechanical characteristics that closely match those of bone, as opposed to those of unmineralised collagen-GAG scaffolds.

The present invention seeks to address at least some of the problems associated with the prior art.

A process for the preparation of a composite biomaterial comprising an inorganic material and an organic material, the process comprising:

(a) providing a first slurry composition comprising a liquid carrier, an inorganic material and an organic material;
(b) providing a mould for the slurry;
(c) depositing the slurry in the mould;
(d) cooling the slurry deposited in the mould to a temperature at which the liquid carrier transforms into a plurality of solid crystals or particles;
(e) removing at least some of the plurality of solid crystals or particles, preferably by sublimation and/or evaporation, to leave a porous composite material comprising an inorganic material and an organic material; and
(f) removing the material from the mould.

The term biomaterial as used herein means a material that is biocompatible with a human or animal body.

The term slurry as used herein encompasses slurries, solutions, suspensions, colloids and dispersions.

The inorganic material will typically comprise a calcium phosphate material.

The organic material will typically comprise a bio-organic species, for example one that can solubilised or suspended in an aqueous medium to form a slurry. Examples include one or more of albumin, glycosaminoglycans, hyaluronan, chitosan, and synthetic polypeptides comprising a portion of the polypeptide sequence of collagen. Collagen is the preferred material, optionally together with a glycosaminoglycan.

The term collagen as used herein encompasses recombinant human (rh) collagen.

In a preferred embodiment, the inorganic material comprises a calcium phosphate material, the organic material comprises collagen and optionally a glycosaminoglycan. This results in a porous composite material comprising the calcium phosphate material and collagen and optionally a glycosaminoglycan. Preferably, the first slurry comprises a co-precipitate of collagen and the calcium phosphate material. More preferably, the first slurry comprises a triple co-precipitate of collagen, a calcium phosphate material and a glycosaminoglycan.

Alternatively, the first slurry may simply comprise a mechanical mixture of collagen and the calcium phosphate material and optionally the glycosaminoglycan. This may be produced by a conventional technique such as described in, for example, EP-A-0164 484 and EP-A-0214070. While a mechanical mixture may be used to form the slurry, a co-precipitate of collagen and the calcium phosphate material or a triple co-precipitate of collagen, the calcium phosphate material and a glycosaminoglycan are preferred.

The calcium phosphate material may be selected, for example, from one or more of brushite, octacalcium phosphate and/or apatite. The calcium phosphate material preferably comprises brushite.

The pH of the slurry is preferably from 2.5 to 6.5, more preferably from 2.5 to 5.5, still more preferably from 3.0 to 4.5, and still more preferably from 3.8 to 4.2.

The slurry composition may comprise one or more glycosaminoglycans. The slurry composition may comprise one or more calcium phosphate materials.

The presence of other species (e.g. silver, silicon, silica, table salt, sugar, etc) in the precursor slurry is not precluded.

At least some of the plurality of solid crystals or particles may be removed by sublimation and/or evaporation to leave a porous composite material comprising collagen, a calcium phosphate material, and optionally a glycosaminoglycan. The preferred method is sublimation.

Steps (d) and (e) may be effected by a freeze-drying technique. If the liquid carrier is water, the sublimation step comprises reducing the pressure in the environment around the mould and frozen slurry to below the triple point of the water/ice/water vapour system, followed by elevation of the temperature to greater than the temperature of the solid-vapor transition temperature at the achieved vacuum pressure. The ice in the product is directly converted into vapor via sublimation as long as the ambient partial liquid vapor pressure is lower than the partial pressure of the frozen liquid at its current temperature. The temperature is typically elevated to at or above 0° C. This step is performed to remove the ice crystals from the frozen slurry via sublimation.

The freeze-drying parameters may be adjusted to control pore size and aspect ratio as desired. In general, slower cooling rates and higher final freezing temperatures (for example, cooling at approximately 0.25° C. per minute to a temperature of about −10° C.) favour large pores with higher aspect ratios, while faster cooling rates and lower final freezing temperatures (for example, cooling at approximately 2.5° C. per minute to a temperature of about −40° C.) favours the formation of small equiaxed pores.

The term “mould” as used herein is intended to encompass any mould, container or substrate capable of shaping, holding or supporting the slurry composition. Thus, the mould in its simplest form could simply comprise a supporting surface. The mould may be any desired shape, and may be fabricated from any suitable material including polymers (such as polysulphone, polypropylene, polyethylene), metals (such as stainless steel, titanium, cobalt chrome), ceramics (such as alumina, zirconia), glass ceramics, and glasses (such as borosilicate glass).

The applicant's earlier application, PCT/GB04/004550, filed 28 Oct. 2004, describes a triple co-precipitate of collagen, brushite and a glycosaminoglycan and a process for its preparation. The content of PCT/GB04/004550 is incorporated herein by reference. A copy of PCT/GB04/004550 is provided in Annex 1.

The process described in PCT/GB04/004550 involves: providing an acidic aqueous solution comprising collagen, a calcium source and a phosphorous source and a glycosaminoglycan; and precipitating the collagen, the brushite and the glycosaminoglycan together from the aqueous solution to form a triple co-precipitate.

The term co-precipitate means precipitation of the two or three compounds where the compounds have been precipitated at substantially the same time from the same solution/dispersion. It is to be distinguished from a material formed from the mechanical mixing of the components, particularly where these components have been precipitated separately, for instance in different solutions. The microstructure of a co-precipitate is substantially different from a material formed from the mechanical mixing of its components.

In the process for preparing the co-precipitate, the calcium source is preferably selected from one or more of calcium nitrate, calcium acetate, calcium chloride, calcium carbonate, calcium alkoxide, calcium hydroxide, calcium silicate, calcium sulphate, calcium gluconate and the calcium salt of heparin. A calcium salt of heparin may be derived from the porcine intestinal mucosa Suitable calcium salts are commercially available, for example, from Sigma-Aldrich Inc. The phosphorus source is preferably selected from one or more of ammonium-dihydrogen phosphate, diammonium hydrogen phosphate, phosphoric acid, disodium hydrogen orthophosphate 2-hydrate (Na₂HPO₄.2H₂O, sometimes termed GPR Sorensen's salt) and trimethyl phosphate, alkali metal salts (eg Na or K) of phosphate, alkaline earth salts (eg Mg or Ca) of phosphate.

Glycosaminoglycans are a family of macromolecules containing long unbranched polysaccharides containing a repeating disaccharide unit. Preferably, the glycosaminoglycan is selected from one or more of chondroitin sulphate, dermatin sulphate, heparin, heparin sulphate, keratin sulphate and hyaluronic acid. Chondroitin sulphate may be chondroitin-4-sulphate or chondroitin-6-sulphate, both of which are commercially available, for example, from Sigma-Aldrich Inc. The chondroitin-6-sulphate may be derived from shark cartilage. Hyaluronic acid may be derived from human umbilical chord. Heparin may be derived from porcine intestinal mucosa.

The collagen may be soluble or insoluble and may be derived from any tissue in any animal and may be extracted using any number of conventional techniques.

Precipitation may be effected by combining the collagen, the calcium source, the phosphorous source and the glycosaminoglycan in an acidic aqueous solution and either allowing the solution to stand until precipitation occurs, agitating the solution, titration using basic titrants such as ammonia, addition of a nucleating agent such as pre-fabricated brushite, varying the rate of addition of the calcium source, or any combination of these or numerous other techniques known in the art.

It will be appreciated that other components may be present in the slurry. For example, growth factors, genes, drugs or other biologically active species may optionally be added, alone or in combination, to the slurry.

In a preferred embodiment, the process according to the present invention advantageously further comprises:

providing a second slurry composition comprising a liquid carrier and an organic material and optionally an inorganic material; and
prior to said cooling step, depositing said second slurry composition in the mould either before or after said first slurry composition has been deposited.

As before, the organic material will typically comprise one or more of collagen (including recombinant human (rh) collagen), a glycosaminoglycan, albumin, hyaluronan, chitosan, and synthetic polypeptides comprising a portion of the polypeptide sequence of collagen.

The second slurry composition may comprise an inorganic material such as, for example, a calcium phosphate material.

Preferably, the second slurry composition comprises a liquid carrier, collagen, optionally a calcium phosphate material, and optionally a glycosaminoglycan. In this embodiment, the second slurry composition preferably comprises a co-precipitate of collagen and a glycosaminoglycan, or a co-precipitate of collagen and a calcium phosphate material, or a triple co-precipitate of collagen, a glycosaminoglycan and a calcium phosphate material. Co-precipitation has already been discussed in relation to the preparation of the first slurry.

Alternatively, the second slurry may simply comprise a mechanical mixture of collagen and optionally one or both of a calcium phosphate material and a glycosaminoglycan. Mechanical mixtures have already been discussed in relation to the preparation of the first slurry.

If present, the calcium phosphate material in the second slurry may be selected from one or more of brushite, octacalcium phosphate and/or apatite.

The first and second slurry compositions will typically be deposited as first and second layers in the mould. For example, the first slurry is deposited in the mould, followed by the second slurry. The mould contents may then be subjected to steps (d), (e) and (f). Accordingly, the process may be used to form a multi-layered material, at least one layer of which preferably comprises a porous composite material comprising collagen, a calcium phosphate material, and optionally a glycosaminoglycan. The layer resulting from the second slurry composition may be a porous or a non-porous layer. If a porous layer is desired, then the pores can be created by sublimation and/or evaporation of a plurality of solid crystals or particles formed in the second slurry. This technique has been already discussed in relation to the first slurry and preferably comprises a freeze drying technique.

The process is carried out in the liquid phase and this is conducive to diffusion between the first slurry layer and the second slurry layer.

The layers may be deposited in any manner of layering orders or geometries. The layers may, for example, be situated vertically (i.e. one on top of the other), horizontally (i.e. one beside the other), and/or radially (one spherical layer on top of the next).

The casting process according to the present invention enables the fabrication of porous monolithic and porous layered scaffolds for use in tissue engineering.

After the first and second slurry compositions have been deposited in the mould, the contents of the mould are preferably left to rest for up to 24 hours before the cooling step. This is advantageous because it allows inter-diffusion of the various slurry constituents between adjacent layers. This results in an improvement in inter-layer bond strength.

The liquid carrier in the first slurry preferably comprises water. The liquid carrier in the second slurry also preferably comprises water.

It will be appreciated that further slurry layers may be deposited in the mould prior to said cooling step, either before or after said first and/or second slurry composition(s) has/have been deposited.

The temperature of the slurry deposited in the mould prior to the cooling step will generally have an effect on the viscosity of the slurry. If the temperature is too high, then this may result in slurries of excessively low viscosity, which may result in complete (and therefore undesirable) intermixing of the first and second layers once the second slurry is deposited. It should also be noted that too high a temperature may result in denaturation of the collagen. On the other hand, too low a temperature may result in slurries with viscosities too high to allow efficient spreading, smoothing or shaping, and may risk the premature formation of ice crystals. Accordingly, the inventors have found that the temperature of the first slurry deposited in the mould prior to the cooling step is preferably in the range of from 2 to 40° C., more preferably from 4 to 37° C., still more preferably from 20 to 37° C. If multiple layered slurry compositions are used, then these ranges are also applicable to the additional slurries.

The step of cooling the first slurry deposited in the mould is preferably carried out to a temperature of ≦0° C. More preferably, the step of cooling is carried out to a temperature in the range of from −100 to 0° C., preferably from −80 to −10° C., more preferably from −40 to −20° C. If multiple layered slurry compositions are used, then these ranges are also applicable to the additional slurries.

The step of cooling the first slurry deposited in the mould is preferably carried out at a cooling rate of 0.02-10° C./min, more preferably from 0.02-6.0° C./min, still more preferably from 0.2-2.7° C./min. If multiple layered slurry compositions are used, then these ranges are also applicable to the additional slurries.

In general, slower cooling rates and higher final freezing temperatures (for example, cooling at 0.25° C. per minute to a temperature of −10° C.) favour large pores with higher aspect ratios, while faster cooling rates and lower final freezing temperatures (for example, cooling at 2.5° C. per minute to a temperature of −40° C.) favours the formation of small equiaxed pores.

The step of cooling the slurry deposited in the mould is preferably carried out at a pressure of from 1-200 kPa, more preferably from 50-150 kPa, still more preferably from 50-101.3 kPa. If multiple layered slurry compositions are used, then these ranges are also applicable to the additional slurries. The inventors have found that pressures below 50 kPa can result in the formation of bubbles within the slurry, while pressures greater than 200 kPa may induce excessive mixing of adjacent layers.

The thickness of the first slurry deposited in the mould is preferably from 0.1-500 mm, more preferably from 0.5-20 mm, still more preferably from 1.0-10 mm. If multiple layered slurry compositions are used, then these ranges are also applicable to the additional slurries. Layers in excess of 500 mm in thickness can be difficult to solidify completely, while layers less than 0.1 mm thick can freeze almost instantaneously, making it difficult to control accurately the progression of ice crystal nucleation and growth.

The viscosity of the first slurry prior to it being deposited in the mould is preferably from 0.1-50 Pa·s, more preferably from 0.1-10 Pa·s, still more preferably from 0.5-5 Pa·s. If multiple layered slurry compositions are used, then these ranges are also applicable to the additional slurries. Slurries with overly high viscosity can be difficult to spread, smooth and shape, while those with excessively low viscosity may result in complete (and therefore undesirable) intermixing of the first and second layers once the second slurry is deposited.

The step of removing at least some of the solid crystals or particles in the first slurry by sublimation is preferably carried out at a pressure of from 0-0.08 kPa, more preferably from 0.0025-0.08 kPa, still more preferably from 0.0025-0.04 kPa. If multiple layered slurry compositions are used, then these ranges are also applicable to the additional slurries. Pressures above that of the triple point of water (approximately 0.08 kPa) can risk the occurrence of melting instead of sublimation, while excessively low pressures are difficult to achieve, and unnecessary for encouraging sublimation.

With regard to the step of removing at least some of the solid crystals or particles in the first slurry by sublimation, if the duration of sublimation is too short, residual water and solvents can cause redissolution of the scaffold walls, thereby compromising the pore architecture. Accordingly, the inventors have found that this step is preferably carried out for up to 96 hours, more preferably from 12-72 hours, still more preferably from 24-36 hours. If multiple layered slurry compositions are used, then these ranges are also applicable to the additional slurries.

The step of removing at least some of the solid crystals or particles in the first slurry by sublimation is preferably carried out at a temperature of from −10-60° C., more preferably from 0-40° C., still more preferably from 20-37° C., still more preferably from 25-37° C. If multiple layered slurry compositions are used, then these ranges are also applicable to the additional slurries. If the temperature during sublimation is too low, the time required until sublimation is complete can become excessively long, while excessively high temperatures (i.e. above 40° C.) can risk denaturation of the collagen.

If the material comprises collagen and a glycosaminoglycan, then the process according to the present invention may further comprise the step of cross-linking the collagen and the glycosaminoglycan in the porous composite biomaterial. Cross-linking will typically take place after the material has been removed from the mould following sublimation. Crosslinking may be effected by subjecting the co-precipitate to one or more of gamma radiation, ultraviolet radiation, a dehyrdothermal treatment, non-enzymatic glycation with a simple sugar such as glucose, mannose, ribose and sucrose, contacting the triple co-precipitate with one or more of glutaraldehyde, carbodiimide (eg ethyl dimethylaminopropyl carbodiimide) and/or nor-dihydroguariaretic acid, or any combination of these methods. These methods are conventional in the art.

If the material comprises calcium phosphate, then the process according to the present invention may further comprise the step of converting at least some of the calcium phosphate material in the porous composite biomaterial to another calcium phosphate phase. For example, the process may comprise the step of converting at least some of the brushite in the porous composite biomaterial to octacalcium phosphate and/or apatite. The conversion of the brushite to octacalcium phosphate and/or apatite is preferably effected by hydrolysation. Phase conversion will typically take place after the material has been removed from the mould (and optionally cross-linked).

Apatite is a class of minerals comprising calcium and phosphate and has the general formula: Ca₅(PO₄)₃(X), wherein X may be an ion that is typically OH⁻, F⁻ and Cl⁻, as well as other ions known to those skilled in the art. The term apatite also includes substituted apatites such as silicon-substituted apatites. The term apatite includes hydroxyapatite, which is a specific example of an apatite. The hydroxyapatite may also be substituted with other species such as, for example, silicon.

As mentioned above, further slurry layers may be deposited in the mould prior to said cooling step, either before or after said first and/or second slurry composition(s) has/have been deposited. The further slurry layers will also typically comprise, for example, a liquid carrier, collagen, optionally a calcium phosphate material, and optionally a glycosaminoglycan. The contents of the mould are preferably left to rest for up to 24 hours before the cooling step so as to allow inter-diffusion of the various slurry constituents between adjacent layers.

Accordingly, the present invention provides a process for the preparation of a composite biomaterial comprising one, two, or more layers. At least one of the layers preferably comprises a porous biocomposite of collagen, a calcium phosphate material, and also preferably a glycosaminoglycan. All of the layers preferably contain collagen.

The composite biomaterial according to the present invention may be used to fabricate, for example, a porous monolithic scaffold, or a multi-layered scaffold in which at least one layer is porous. The composite biomaterial according to the present invention is advantageously used as a tissue regeneration scaffold for musculoskeletal and dental applications.

The process according to the present invention preferably involves incorporating collagen as an organic constituent in the first and second layers (collagen is preferably the major organic constituent in the first and second layers). If additional layers are present, then the process preferably involves incorporating collagen as an organic constituent in one or more of these further layers (collagen is also preferably the major organic constituent in the one or more further layers). The process involves fabricating all layers, and thus the interfaces between them, simultaneously in the liquid phase. This results in the creation of a strong interface between the layers through inter-diffusion. The term inter-diffusion refers to mixing that occurs as a result of molecular diffusion or Brownian motion when two slurries of differing composition are placed in integral contact.

In a second aspect, the present invention provides a synthetic composite biomaterial, wherein at least part of the biomaterial is formed from a porous co-precipitate comprising a calcium phosphate material and one or more of collagen (including recombinant human (rh) collagen), a glycosaminoglycan, albumin, hyaluronan, chitosan or a synthetic polypeptides comprising a portion of the polypeptide sequence of collagen, wherein the macropore size range (pore diameter) is preferably from 1-1000 microns, more preferably from 200-600 microns. The material preferably comprises collagen. The calcium phosphate material is preferably selected from one or more of brushite, octacalcium phosphate and/or apatite. The porous material preferably comprises a co-precipitate of the collagen and the calcium phosphate material. This has already been described in relation to the first aspect of the invention.

The term porous as used herein means that the material may contain macropores and/or micropores. Macroporosity typically refers to features associated with pores on the scale of greater than approximately 10 microns. Microporosity typically refers to features associated with pores on the scale of less than approximately 10 microns. It will be appreciated that there can be any combination of open and closed cells within the material. For example, the material will generally contain both macropores and micropores. The macroporosity is generally open-celled, although there may be a closed cell component.

The macropore size range (pore diameter) in the porous material according to the second aspect of the present invention is typically from 1 to 1200 microns, preferably from 10 to 1000 microns, more preferably from 100 to 800 microns, still more preferably from 200 to 600 microns.

The mean aspect ratio range in the porous material according to the second aspect of the present invention is preferably from 1 to 50, more preferably from 1 to 10, and most preferably approximately 1.

The pore size distribution (the standard deviation of the mean pore diameter) in the porous material according to the second aspect of the present invention is preferably from 1 to 800 microns, more preferably from 10 to 400 microns, and still more preferably from 20 to 200 microns.

The porosity in the porous material according to the second aspect of the present invention is preferably from 50 to 99.99 vol %, and more preferably from 70 to 98 vol %.

The percentage of open-cell porosity (measured as a percentage of the total number of pores both open- and closed-cell) in the porous material according to the second aspect of the present invention is preferably from 1 to 100%, more preferably from 20 to 100%, and still more preferably from 90 to 100%.

In a third aspect, the present invention provides a synthetic composite biomaterial, wherein at least part of the biomaterial is formed from a porous material comprising a calcium phosphate material and two or more of collagen (including recombinant human (rh) collagen), a glycosaminoglycan, albumin, hyaluronan, chitosan and a synthetic polypeptides comprising a portion of the polypeptide sequence of collagen. The material preferably comprises collagen and a glycosaminoglycan. The calcium phosphate material is preferably selected from one or more of brushite, octacalcium phosphate and/or apatite. The porous material preferably comprises a triple co-precipitate of collagen, a glycosaminoglycan and the calcium phosphate material. This has already been described in relation to the first aspect of the invention. The macropore size range (pore diameter) in the porous material according to the second aspect of the present invention is also applicable to the third aspect. The same is true for the mean aspect ratio range, the pore size distribution, the porosity and the percentage of open-cell porosity.

In a fourth aspect, the present invention provides a synthetic composite biomaterial comprising:

a first layer formed of a composite biomaterial according to the second or third aspect of the present invention; and
a second layer joined to the first layer and formed of a material comprising collagen, or a co-precipitate of collagen and a glycosaminoglycan, or a co-precipitate of collagen and a calcium phosphate material, or a triple co-precipitate of collagen, a glycosaminoglycan and a calcium phosphate material. The calcium phosphate material is preferably selected from one or more of brushite, octacalcium phosphate and/or apatite.

The first and second layers are preferably integrally formed. Advantageously, this may be achieved by a process involving liquid phase co-synthesis. This encompasses any process in which adjacent layers, either dense or porous, of a material comprising multiple layers are formed by placing the slurries comprising the precursors to each layer in integral contact with each other before removal of the liquid carrier or carriers from said slurries, and in which removal of said liquid carrier or carriers from all layers is preferably performed at substantially the same time. Placing the precursor slurries in integral contact before removal of the liquid carrier (i.e. while still in the liquid phase) allows interdiffusion to occur between adjacent slurries. This results in a zone of interdiffusion at the interface between adjacent layers of the resulting material, within which the material composition is intermediate to the material compositions of the adjacent layers. The existence of a zone of interdiffusion can impart mechanical strength and stability to the interface between adjacent layers. Accordingly, the first and second layers are preferably joined to one another through an inter-diffusion layer.

Alternatively, the first and second layers may be joined to one another through an inter-layer. The term inter-layer refers to any layer deposited independently between two other layers for the purpose of improving inter-layer bond strength or blocking the passage of cells, molecules or fluids between adjacent layers of the resulting scaffold, and may, for example, contain collagen, glycosaminoglycans, fibrin, anti-angiogenic drugs (e.g. suramin), growth factors, genes or any other constituents. An inter-layer is distinguished from an inter-diffusion layer by the fact that an inter-layer is deposited separately as a slurry whose composition is distinct from the composition of its adjacent layers, while an inter-diffusion layer is formed exclusively as a result of inter-diffusion between adjacent layers.

The first layer is porous. The second layer is also preferably porous, although it can be non-porous or substantially non-porous layer if desired.

The macropore size range (pore diameter) in the porous material according to the second aspect of the present invention is also applicable to the first and/or second layers in the embodiment according to the fourth aspect. The same is true for the mean aspect ratio range, the pore size distribution, the porosity and the percentage of open-cell porosity.

In any of the second, third and fourth aspects, the biomaterial may comprise one or more further layers joined to the first and/or second layers, each of said further layers preferably being formed of a material comprising collagen, or a co-precipitate of collagen and a glycosaminoglycan, or a co-precipitate of collagen and a calcium phosphate material, or a triple co-precipitate of collagen, a glycosaminoglycan, and a calcium phosphate material. The calcium phosphate material is preferably selected from one or more of brushite, octacalcium phosphate and/or apatite. The first and second layers and said one or more further layers are preferably integrally formed, and adjacent layers are preferably joined to one another through an inter-diffusion layer, which is typically formed by liquid phase co-synthesis. Generally, at least one of said further layers will be porous. Again, the macropore size range (pore diameter) in the porous material according to the second aspect of the present invention is also applicable to one or more of these further layers. The same is true for the mean aspect ratio range, the pore size distribution, the porosity and the percentage of open-cell porosity.

Differences in pore sizes between adjacent layers may vary from almost negligible to as great as +/−1000 microns.

Unless otherwise stated, the following description is applicable to any aspect of the present invention.

If the material comprises collagen and a glycosaminoglycan, then the collagen and the glycosaminoglycan may be crosslinked.

The collagen is preferably present in the material in an amount of from 1 to 99 wt %, preferably from 5 to 90 wt %, more preferably from 15 to 60 wt %.

The glycosaminoglycan is preferably present in the material in an amount of from 0.01 to 20 wt %, more preferably from 1 to 12 wt %, still more preferably from 1 to 5.5 wt %.

If the material comprises brushite, then the ratio of collagen to brushite is preferably from 10:1 to 1:100 by weight, more preferably from 5:1 to 1:20 by weight.

If the material comprises octacalcium phosphate, then the ratio of collagen to octacalcium phosphate is preferably 10:1 to 1:100 by weight, more preferably from 5:1 to 1:20 by weight.

The ratio of collagen to the glycosaminoglycan is preferably from 8:1 to 30:1 by weight.

The biomaterial according to the present invention may be used as a substitute bone or dental material. Accordingly, the present invention provides a synthetic bone material, bone implant, bone graft, bone substitute, bone scaffold, filler, coating or cement comprising a biomaterial as herein described.

The biomaterial is advantageously provided in the form of a multi-layered scaffold. In particular, the present invention provides tissue regeneration scaffolds for musculoskeletal and dental applications. Multilayer (i.e. two or more layers) scaffolds according to the present invention may find application in, for example, bone/cartilage interfaces (eg articular joints), bone/tendon interfaces (eg tendon insertion points), bone/ligament interfaces (eg ligament insertion points), and tooth/ligament interfaces (eg tooth/periodontal ligament juncture).

Although the present invention is primarily concerned with scaffolds for tissue engineering applications, the material according to the present invention may be used to fabricate implants that persist in the body for quite some time. For example, a semi-permanent implant may be necessary for tendon and ligament applications.

The present invention further provides a porous composite biomaterial obtainable by a process as herein described.

### Synthesis Method

The present invention will now be described further by way of example. The preferred method of synthesis comprises a sequence of steps, which can be applied in whole or in part, to produce porous scaffolds having one or more layers at least one of which preferably comprises a triple co-precipitate of collagen, a glycosaminoglycan and a calcium phosphate material.

### Step 0: Slurry Preparation

The preparation of mineralised collagen/GAG/brushite slurry or slurries may be achieved using the method outlined in the applicant's earlier patent application, PCT/GB04/004550, filed 28 Oct. 2004. The content of PCT/GB04/004550 is incorporated herein by reference. A copy of PCT/GB04/004550 is provided in Annex 1.

The preparation of unmineralised collagen/GAG slurry or slurries may be achieved using a method as outlined in Yannas et al., 1989; O'Brien et al., 2004; O'Brien et al., 2005); Loree et al., (1989).

Growth factors, genes, drugs or other biologically active species may optionally be added, alone or in combination, to the slurry via mechanical mixing at this stage to facilitate their incorporation into the scaffold. In the case of scaffolds with more than one layer, the biologically active species incorporated into one layer need not be the same as the species incorporated into the next.

### Step I: Casting

Step I-a: Casting of 1st layer
Step I-b: Casting of 2nd layer
Step I-c: Casting of 3rd layer
Step I-n: Casting of nth layer

The casting step(s) involve the successive deposition of a slurry or slurries, in solution, suspension, colloid, or dispersion form, where water comprises the major diluent, into a mould, in which at least one of the slurries comprises a triple co-precipitate of collagen, one or more glycosaminoglycans and the calcium phosphate brushite, and all slurries contain collagen.

The mould may be any desired shape, and may be fabricated of any of a number of materials including polymers (such as polysulphone, polypropylene, polyethylene), metals (such as stainless steel, titanium, cobalt chrome) or ceramics (such as alumina, zirconia), glass ceramics, or glasses (such as borosilicate glass).

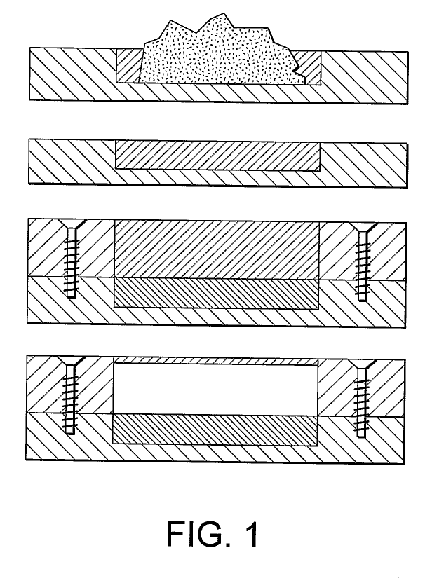

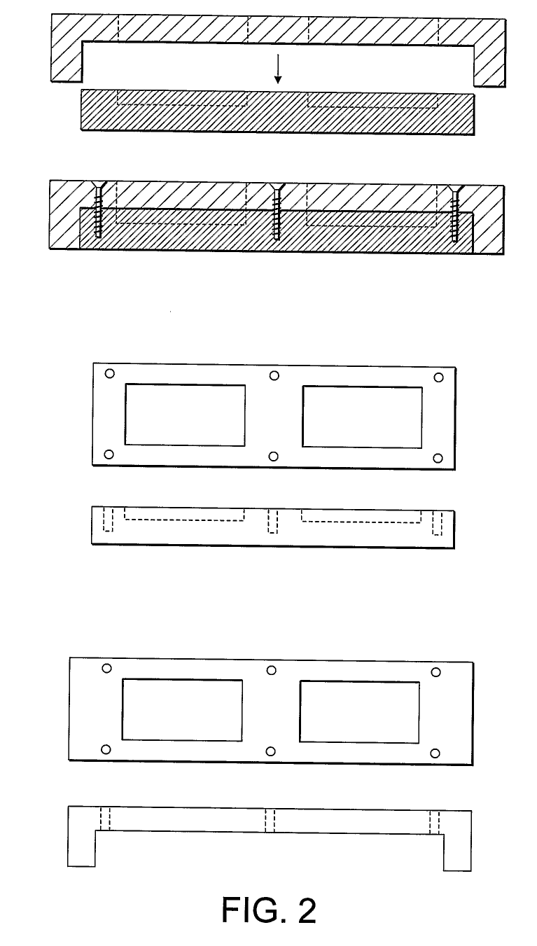

The mould may be constructed specifically to facilitate layering. Examples of suitable designs are shown in FIGS. 1 and 2.

The layers may, for example, be situated vertically (i.e. one on top of the other), horizontally (i.e. one beside the other), and/or radially (one spherical layer on top of the next).

In the event that the scaffold comprises one layer, the single layer to be cast comprises a slurry of a co-precipitate comprising collagen, a calcium phosphate material, which is preferably brushite, and optionally a glycosaminoglycan. Preferably, the slurry comprises a triple co-precipitate comprising collagen, brushite and a glycosaminoglycan. The preferred thickness of the layer is specified in the appropriate section of Table 1.

In the event that the scaffold comprises two layers, at least one of the layers to be cast comprises a slurry of a co-precipitate comprising collagen, a calcium phosphate material, which is preferably brushite, and optionally a glycosaminoglycan. Preferably, the slurry comprises a triple co-precipitate comprising collagen, brushite, and a glycosaminoglycan. The preferred thickness of this layer is specified in the appropriate section of Table 1. The other layer comprises a slurry comprising collagen, optionally a calcium phosphate material, and optionally a glycosaminoglycan. This slurry composition preferably comprises a co-precipitate of collagen and a glycosaminoglycan, a co-precipitate of collagen and a calcium phosphate material such as brushite, or a triple co-precipitate of collagen, a glycosaminoglycan and a calcium phosphate material, which is preferably brushite.

Further layers may be included as desired and these further layers are preferably formed from a slurry comprising collagen, optionally a calcium phosphate material, and optionally a glycosaminoglycan. The further slurry compositions preferably comprise a co-precipitate of collagen and a glycosaminoglycan, a co-precipitate of collagen and a calcium phosphate material such as brushite, or a triple co-precipitate of collagen, a glycosaminoglycan and a calcium phosphate material, which is preferably brushite.

The composition of the slurries in each subsequent layer may be identical, vary slightly, or vary significantly, provided that collagen and preferably also a glycosaminoglycan are present in each layer, and that at least one of the layers also contains a calcium phosphate material such as, for example, brushite.

### Step II: Inter-Diffusion

The co-diffusion step involves allowing the respective layers of the cast, layered slurry to inter-diffuse. This step is performed for the purpose of allowing inter-diffusion of slurry constituents between adjacent layers, thereby increasing the inter-layer bond strength after solidification and sublimation. Preferred conditions for the inter-diffusion step are listed in the appropriate section of Table 2.

### Step III: Controlled Cooling

The controlled cooling step involves placing the mould containing the slurry in an environment, which is then cooled at a controlled rate to a final temperature less than 0° C. This step is performed to initiate and control the rate of ice crystal nucleation and growth within the slurry. Ice crystals are then subsequently removed by sublimation leaving a porous scaffold. The architecture of the ice crystal network will determine the ultimate pore structure of the scaffold. The preferred parameters for cooling are listed in Table 3.

### Step IV: Annealing

The annealing step involves allowing the slurry to remain at the final temperature of the controlled cooling step for a designated amount of time. This step is performed to ensure that the slurry freezes completely or substantially completely. The preferred parameters for annealing are listed in Table 4.

### Step V: Sublimation

The sublimation step comprises reducing, while the frozen slurry is maintained at roughly the final temperature of the controlled cooling and annealing steps, the pressure in the environment around the mould and frozen slurry to below the triple point of the water/ice/water vapour system, followed by elevation of the temperature to greater than the temperature of the solid-vapor transition temperature at the achieved vacuum pressure (typically ≧0° C.). This step is performed to remove the ice crystals from the frozen slurry via sublimation. The advantage of sublimation over evaporation as a means of water removal is that it leaves a network of empty space (i.e. pores) that mimics precisely the architecture of the previously existing network of ice crystals. If the ice is allowed to melt, the ice crystal network loses its shape, and the architecture of the resulting pore network is compromised. Preferred parameters for the sublimation step are shown in Table 5.

### Step V+I: Crosslinking

If desired, the process may also involve a crosslinking step to crosslink the collagen and the glycosaminoglycan. This is described in the applicant's earlier patent application, PCT/GB04/004550, filed 28 Oct. 2004. The content of PCT/GB04/004550 is incorporated herein by reference. A copy of PCT/GB04/004550 is provided in Annex 1.

### EXAMPLES

### Example I

### Single-Layer Scaffold of Collagen/GAG/CaP

### Materials

Collagen: Type I, microfibrillar collagen from bovine tendon, Integra Life Sciences Plainsboro, N.J., USA
GAG: Chondroitin-6-sulphate from shark cartilage, sodium salt, Sigma-Aldrich Inc (St. Louis, Mo., USA)
Calcium Sources: (i) Calcium hydroxide (Ca(OH)₂), Sigma-Aldrich Inc (St. Louis, Mo., USA); (ii) Calcium nitrate (Ca(NO₃)₂.4H₂O), Sigma-Aldrich Inc (St. Louis, Mo., USA)
Phosphorous Source: Orthophosphoric acid (H₃PO₄), BDH Laboratory Supplies (Poole, United Kingdom)
Crosslinking Agents: 1-Ethyl-3-(3-Dimethylaminopropyl) Carbodiimide (=EDAC), Sigma-Aldrich Inc (St. Louis, Mo., USA); N-Hydroxysuccinimide (=NHS), Sigma-Aldrich Inc (St. Louis, Mo., USA)

### Procedure

### Step 0: Slurry Preparation

3.8644 g collagen was dispersed in 171.4 mL of 0.1383M H₃PO₄ cooled in an ice bath by blending for 90 minutes at 15,000 rpm using a homogeniser equipped with a 19 mm diameter stator to create a highly viscous collagen dispersion. In parallel, 0.3436 g chondroitin-6-sulphate (GAG) was allowed to dissolve in 14.3 mL of 0.1383M H₃PO₄ at room temperature by shaking periodically to disperse dissolving GAG in order to produce a GAG solution. After 90 minutes, the 14.3 mL of GAG solution was added to the mixing collagen dispersion at a rate of approximately 0.5 mL/min under continuous homogenisation at 15,000 rpm, and the resulting highly-viscous collagen/GAG dispersion blended for an additional 90 minutes. After 90 minutes of mixing, 1.804 g Ca(OH)₂ and 0.780 g Ca(NO₃)₂.4H₂O were added to the highly-viscous collagen/GAG dispersion over 30 minutes under constant blending at 15,000 rpm, creating a collagen/GAG/CaP slurry, the pH of which was approximately 4.0. The collagen/GAG/CaP slurry was allowed to remain at 25° C. for a period of 48 hours mixing on a stir plate, and was then placed at 4° C. for a subsequent 12 hours. The chilled slurry was then degassed in a vacuum flask over 25 hours at a pressure of 25 Pa.

### Step I: Casting

15 mL of the mineralised collagen/GAG/CaP slurry was cast into a polysulphone mould, 50 mm long by 30 mm wide by 10 mm deep, using an auto-pipettor. All large bubbles were removed from the slurry using a hand pipettor.

### Step II: Inter-Diffusion

As the scaffold for Example I comprised only one layer, the inter-diffusion step was unnecessary.

### Step III: Controlled Cooling

The mould and slurry were placed in a VirTis Genesis freeze dryer (equipped with temperature-controlled, stainless steel shelves) and the shelf temperature of the freeze dryer ramped from 4° C. to −20° C. at a rate of approximately 2.4° C. per minute.

### Step IV: Annealing

The shelf temperature of the freeze dryer was maintained at −20° C. for 10 hours.

### Step V: Sublimation

While still at a shelf temperature of −20° C., a vacuum of below 25 Pa (approximately 200 mTorr) was applied to the chamber containing the mould and the (now frozen) slurry. The temperature of the chamber was then raised to 37° C., and sublimation allowed to continue for 36 hours. The vacuum was then removed, and the temperature returned to room temperature, leaving a single-layered scaffold of collagen/GAG/CaP, 50 mm by 30 mm by 10 mm in size.

### Step V+I: Crosslinking

Scaffolds were hydrated in 40 mL deionised water for 20 minutes. 20 mL of a solution of 0.035M EDAC and 0.014M NHS was added to the container containing the scaffolds and deionised water, and the scaffolds were allowed to crosslink for 2 hours at room temperature under gentle agitation. The EDAC solution was removed, and the scaffolds were rinsed with phosphate buffer solution (PBS) and then allowed to incubate at 37° C. for 2 hours in fresh PBS under mild agitation. After two hours in PBS, the scaffolds were rinsed by allowing them to incubate in deionised water for two ten-minute intervals at 37° C. under mild agitation. The scaffolds were then freeze-dried to remove any residual water by controlled cooling from room temperature to −20° C. at a rate of approximately 2.4° C. per minute, followed by annealing at −20° C. for approximately 5 hours, and then sublimation at below 25 Pa at 37° C., resulting in a crosslinked collagen/GAG/CaP scaffold roughly 50 mm by 30 mm by 10 mm in size.

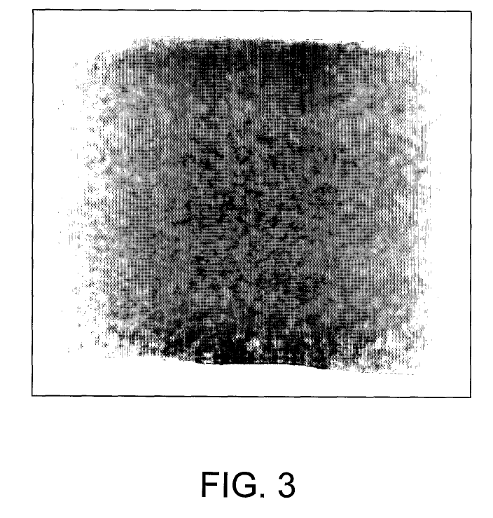

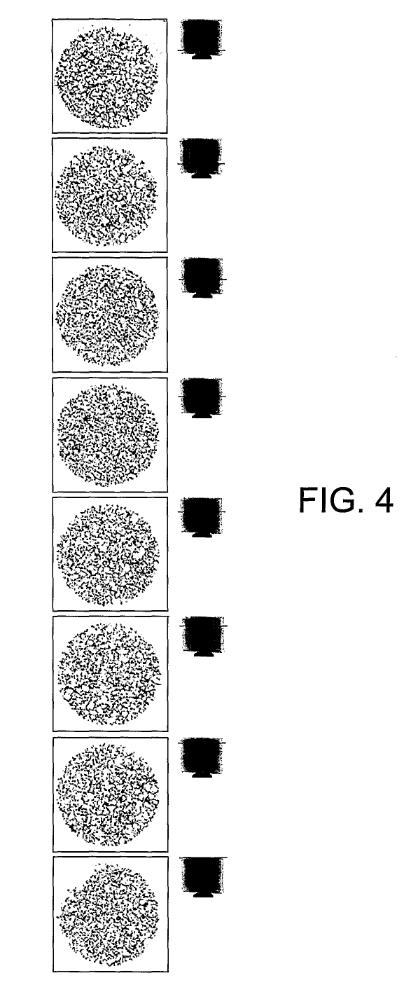

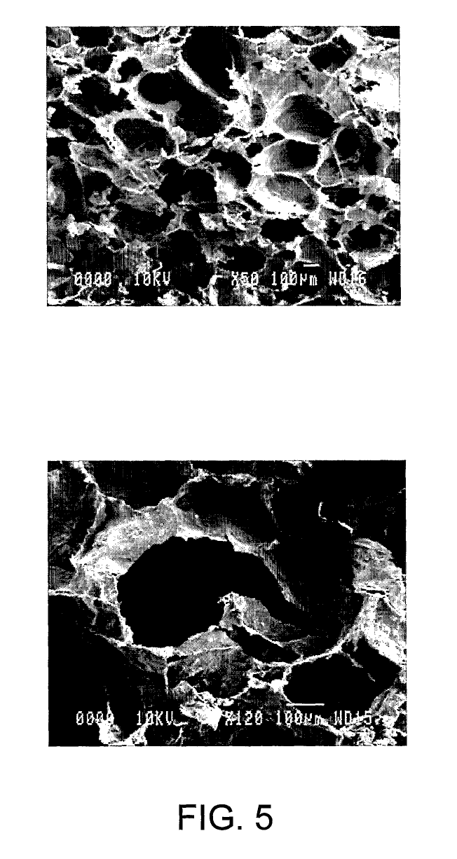

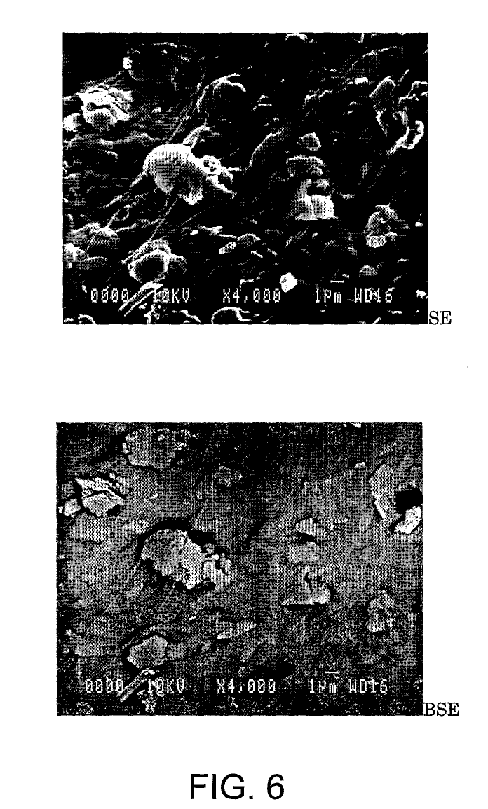

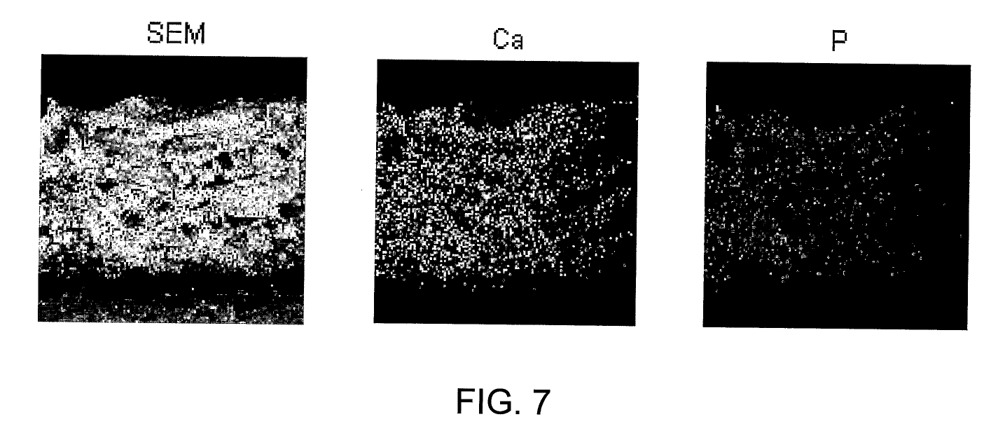

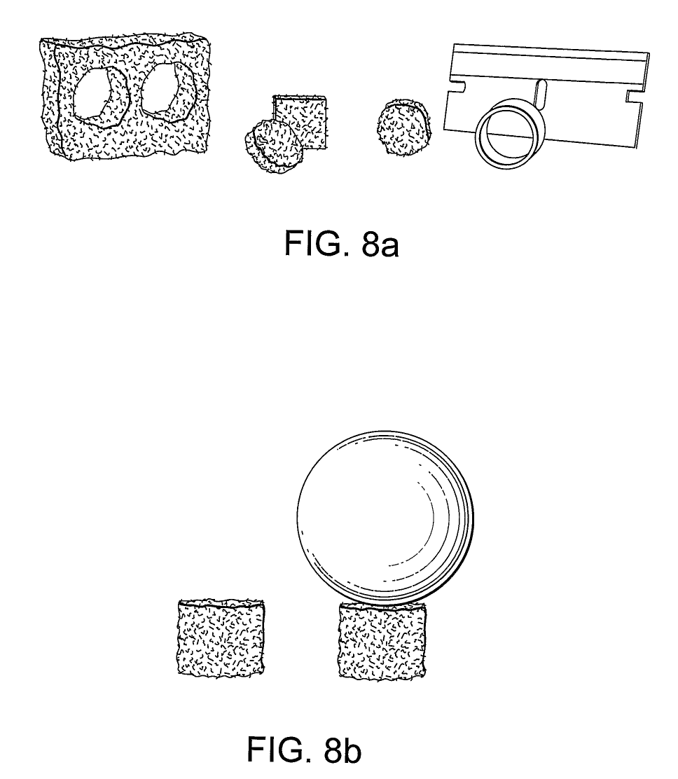

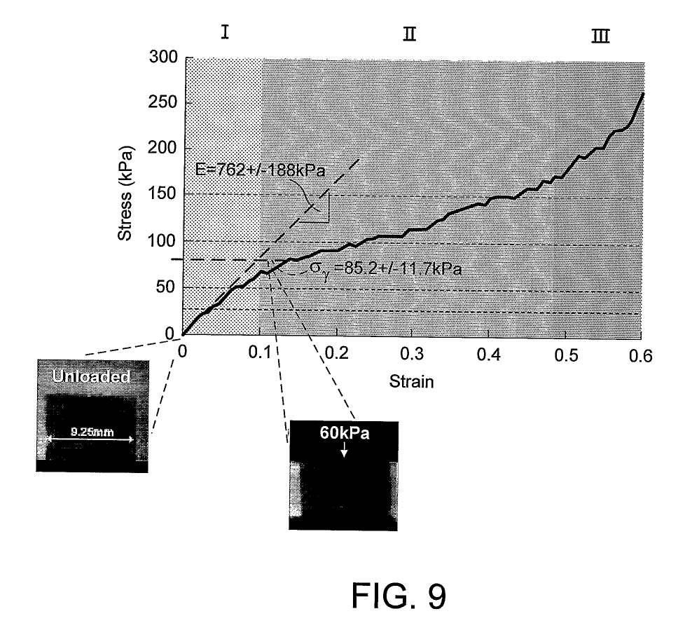

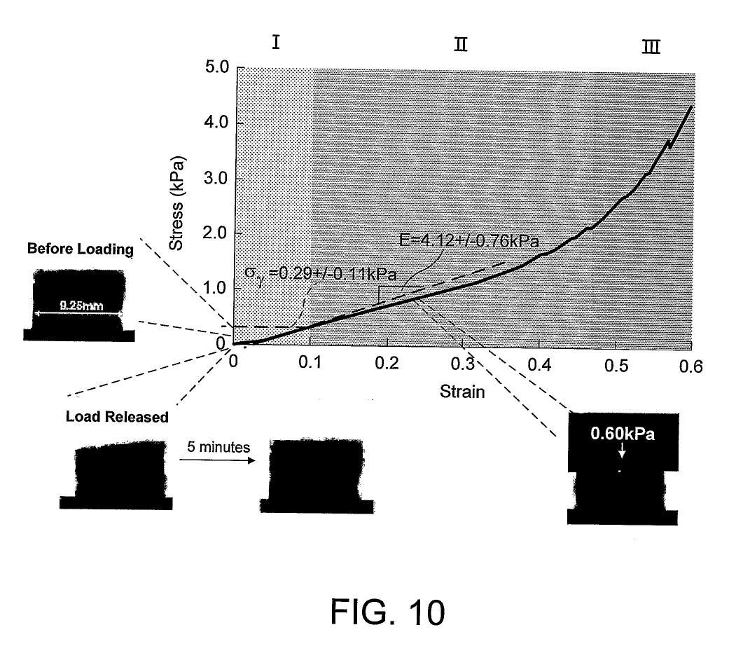

X-ray microtomographic images, scanning electron microscope images, ion distribution maps and compressive mechanical behaviour of the resulting one-layer scaffolds are shown in FIGS. 3 to 10. FIG. 3 shows a profile of a 9.5 mm×9.5 mm cylindrical section of the scaffold produced by the above procedure, as viewed through X-ray microtomography. Of note is the substantially uniform nature of both material composition and porosity throughout the scaffold. Sequential cross-sections of the same scaffold are shown in FIG. 4, again illustrating the uniform nature of the scaffold pore structure; also evident in FIG. 4 is the high degree of pore interconnectivity, the equiaxed pore morphology and the large (mean diameter of 500 microns) macropore size. In FIG. 5, SEM micrographs again show the macropore morphology while also showing the presence of limited microporosity, visible within the walls of certain macropores. High (4000×) magnification secondary (i.e. topography-sensitive) and backscattered (i.e. composition-sensitive) electron images of a region of the scaffold wall (FIG. 6) demonstrate the compositional homogeneity of the scaffold walls, despite the presence of limited topological variations in the form of protruding nodules approximately 1-2 microns in size. The calcium and phosphorous maps shown in FIG. 7 corroborate the conclusion of substantially compositional homogeneity throughout the scaffold, with both elements distributed evenly throughout the scaffold. FIG. 8 shows single-layered scaffolds in the dry state, and illustrates their ability to be cut to any desired shape without crumbling, cracking or losing their integrity using common surgical tools such as scalpels, razor blades and trephine blades (circular cutting tools used during corneal transplantation); FIG. 8 also illustrates the weight bearing capacity of dry single-layered scaffolds under the weight of a solid-steel ball-bearing. In FIG. 9, the behaviour of single-layered scaffolds in the dry state is shown. This behaviour exhibits the three-stages of deformation typical of porous solids, with an elastic modulus of 762+/−188 kPa and a compressive yield stress of 85.2+/−11.7 kPa. It is significant to note that the yield strength of the dry scaffolds allows them to withstand firm thumb pressure (during insertion into a defect site, for example) without deforming permanently yet still be formed when strong thumb pressure is applied (by a surgeon modifying the shape of the implant, for example). In FIG. 10, the compressive deformation of single-layered scaffolds in the hydrated state is shown. As in the dry state, hydrated mineralised collagen/GAG scaffolds exhibit three-stage mechanical behaviour under compressive loading, but with elastic modulus (4.12+/−0.76 kPa) and yield stress (0.29+/−0.11 kPa) roughly an order of magnitude lower than the corresponding properties of dry scaffolds. Furthermore, evidence of viscoelastic strain recovery has been observed following release of compressive stresses in the collapse plateau region.

### Example II

### Two-Layer Mineralised-Unmineralised Scaffold

### Materials

Collagen (for mineralised slurry): Type I microfibrillar collagen from bovine tendon, Integra Life Sciences Plainsboro, N.J., USA
GAG (for mineralised slurry): Chondroitin-6-sulphate from shark cartilage, sodium salt, Sigma-Aldrich Inc (St. Louis, Mo., USA)

### Type II Collagen

+GAG (for unmineralised slurry): Type II Collagen and GAG (Collagen/GAG) slurry solubilised from porcine cartilage, Gelstlich Biomaterials (Wolhusen, Switzerland).
Calcium Sources: (i) Calcium hydroxide (Ca(OH)₂) Sigma-Aldrich Inc (St. Louis, Mo., USA); (ii) Calcium nitrate, Ca(NO₃)₂.4H₂O, Sigma-Aldrich Inc (St. Louis, Mo., USA)
Phosphorous Source: Orthophosphoric acid (H₃PO₄), BDH Laboratory Supplies (Poole, United Kingdom)
Crosslinking Agents: 1-Ethyl-3-(3-Dimethylaminopropyl) Carbodiimide (=EDAC), Sigma-Aldrich Inc (St. Louis, Mo., USA); N-Hydroxysuccinimide (=NHS), Sigma-Aldrich Inc (St. Louis, Mo., USA)

### Step 0: Slurry Preparation

### Mineralised Slurry Preparation

3.8644 g collagen was dispersed in 171.4 mL of 0.1383M H₃PO₄ cooled in an ice bath by blending for 90 minutes at 15,000 rpm using a homogeniser equipped with a 19 mm diameter stator to create a highly viscous collagen dispersion. In parallel, 0.3436 g chondroitin-6-sulphate (GAG) was allowed to dissolve in 14.3 mL of 0.1383M H₃PO₄ at room temperature by shaking periodically to disperse dissolving GAG in order to produce a GAG solution. After 90 minutes, the 14.3 mL of GAG solution was added to the mixing collagen dispersion at a rate of approximately 0.5 mL/min, under continuous homogenisation at 15,000 rpm, and the resulting highly-viscous collagen/GAG dispersion blended for an additional 90 minutes. After 90 minutes of mixing, 1.804 g Ca(OH)₂ and 0.780 g Ca(NO₃)₂.4H₂O were added to the highly-viscous collagen/GAG dispersion over 30 minutes under constant blending at 15,000 rpm, creating a collagen/GAG/CaP slurry, the pH of which was approximately 4.0. The chilled slurry was then degassed in a vacuum flask over 25 hours at a pressure of 25 Pa, reblended using the homogenizer over 30 minutes, and then degassed again for 48 hours.

### Unmineralised Slurry Preparation

Type II collagen/GAG slurry was removed from refrigerator and allowed to return to room temperature.

### Step I: Casting

2.5 mL of the unmineralised Type II collagen/GAG slurry was placed in the bottom portion of a combination polysulphone mould, the bottom portion of which measured 50 mm in length by 30 mm in width by 2 mm in depth. The slurry was smoothed to a flat surface using a razor blade. An upper collar, also made of polysulphone, and measuring 50 mm in length by 30 mm in width by 6 mm in depth, was attached to the bottom portion of the mould containing the smoothed, unmineralised slurry. 9 mL of the mineralised collagen/GAG/CaP slurry was placed, in an evenly distributed manner, on top of the smoothed, unmineralised layer and within the previously empty upper collar. All large bubbles were removed from the slurry using a hand pipettor.

### Step II: Inter-Diffusion

The layered slurry was allowed to remain at room temperature and pressure for a total of 4 hours, before being placed in the freeze dryer.

### Step III: Controlled Cooling

The mould and layered slurry were placed in a VirTis Genesis freeze dryer (equipped with temperature-controlled, stainless steel shelves) and the shelf temperature of the freeze dryer ramped from 4° C. to −40° C. at a rate of approximately −2.4° C. per minute.

### Step IV: Annealing

The shelf temperature of the freeze dryer was maintained at −40° C. for 10 hours.

### Step V: Sublimation

While still at a shelf temperature of −40° C., a vacuum of below 25 Pa (approximately 200 mTorr) was applied to the chamber containing the mould and the (now frozen) layered slurry. The temperature of the chamber was then raised to 37° C., and sublimation allowed to continue for 36 hours. The vacuum was then removed, and the temperature returned to room temperature, leaving a two-layered scaffold of collagen/GAG/CaP, 50 mm by 30 mm by 8 mm in size, comprised of an unmineralised layer 2 mm thick, and a mineralised layer 6 mm thick.

### Step V+I: Crosslinking

Scaffolds were hydrated in 32 mL deionised water for 20 minutes. 18 mL of a solution of 0.035M EDAC and 0.014M NHS was added to the container containing the scaffolds and deionised water, and the scaffolds were allowed to crosslink for 2 hours at room temperature under gentle agitation. The EDAC solution was removed and the scaffolds were then rinsed with phosphate buffer solution (PBS) and then allowed to incubate at 37° C. for 2 hours in fresh PBS under mild agitation. After two hours in PBS, the scaffolds were rinsed by allowing them to incubate in deionised water for two 10-minute intervals at 37° C. under mild agitation. The scaffolds were then freeze-dried to remove any residual water by controlled cooling from room temperature to −20° C. at a rate of approximately −2.4° C. per minute, followed by annealing at −20° C. for 5 hours, and finally by sublimation at below 25 Pa at 37° C. for 24 hours, resulting in a crosslinked, layered collagen/GAG/CaP scaffold roughly 50 mm by 30 mm by 8 mm in size, comprised of an unmineralised layer 2 mm thick, and a mineralised layer 6 mm thick.

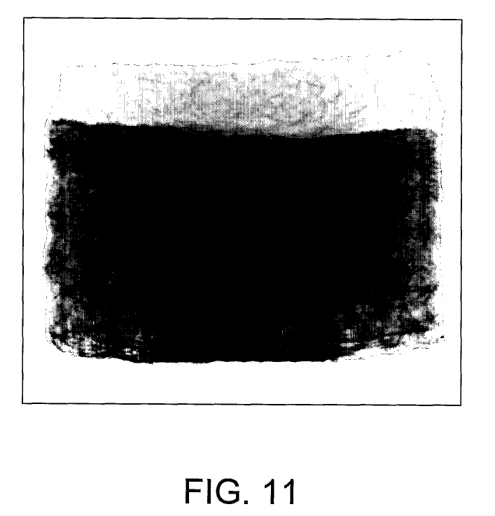

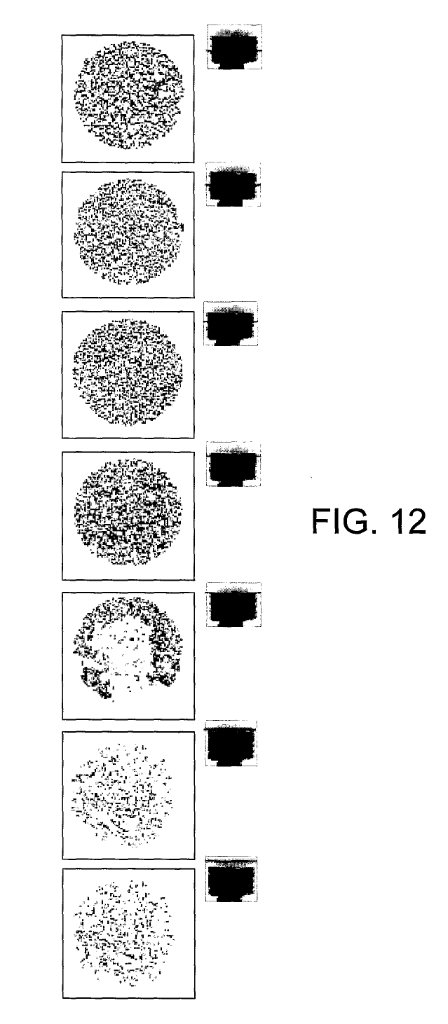

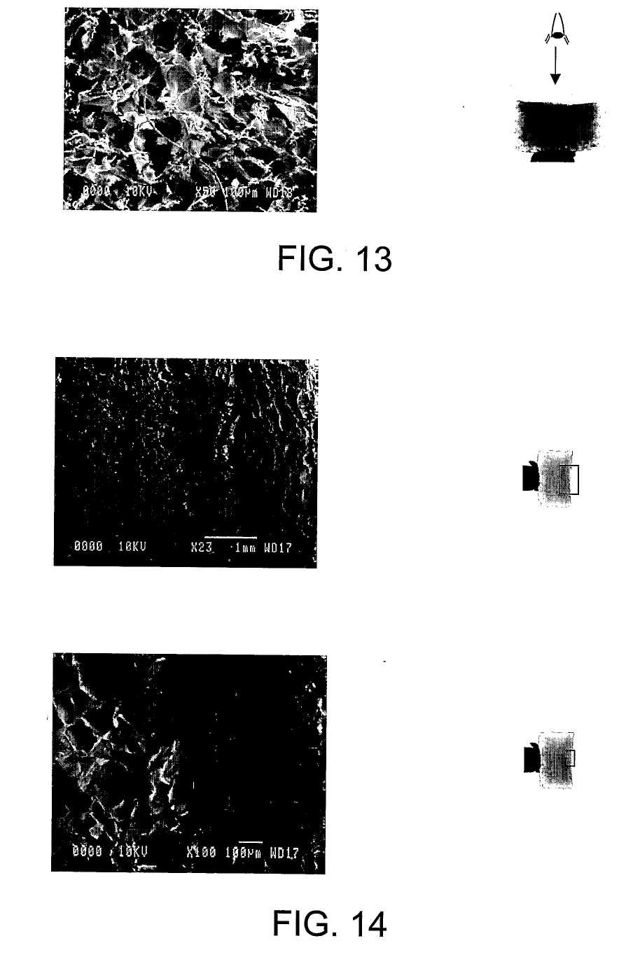

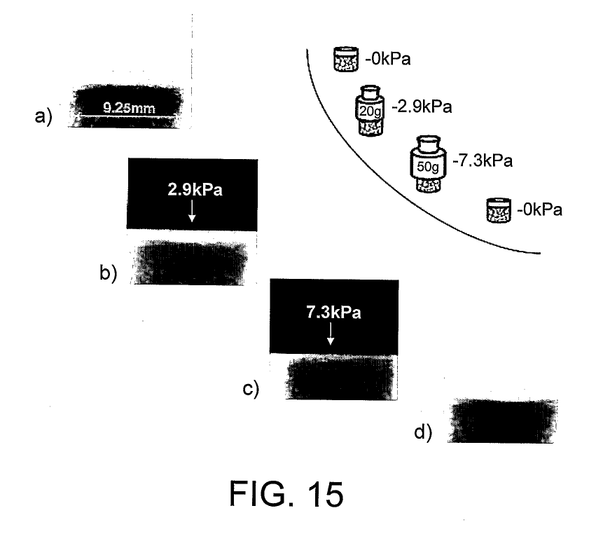

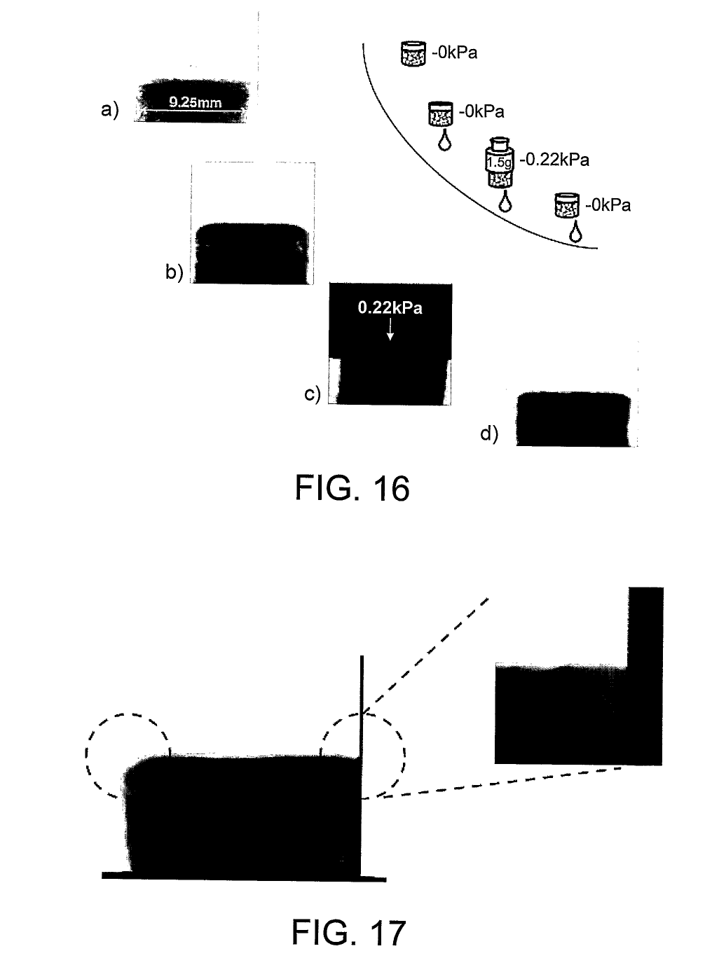

X-ray microtomographic images, scanning electron microscope images, and ion distribution maps of the resulting two-layer scaffolds are shown in FIGS. 11 to 17. An x-ray microtomographic image of a 9.5 mm×9.5 mm cylindrical section of the two-layer scaffold produced by the procedure described above is shown in FIG. 11. The opaque lower region shows the mineralised layer, while the more translucent upper region represents the unmineralised layer. It can be seen that both layers are largely uniform, both in terms of porosity and composition. The serial cross sections shown in FIG. 12 show the mean macropore size in the mineralised layer to be approximately 400 microns, while that in the unmineralised layer is on the order of 700 microns; the pores in both mineralised and unmineralised layers exhibit an equiaxed morphology. The SEM image in FIG. 13 shows a top view of the unmineralised layer, illustrating that little evidence of microporosity is present, while the images of the interface region shown in FIG. 14 demonstrate the lack of any large voids or other discontinuities separating the mineralised and unmineralised layers. In FIG. 15 the behaviour of two-layered scaffolds under compressive loading is shown. Upon application of compressive load, the compliant unmineralised layer begins to compress, resulting in near-complete compaction of the cartilaginous compartment at stresses insufficient to induce any significant deformation in mineralised scaffolds. After the load is released, the unmineralised collagen/GAG layer returns to its original shape almost instantaneously (FIG. 15d). FIG. 16 illustrates the mechanical behaviour of two-layered scaffolds in the hydrated state. Once hydrated, the unmineralised collagen/GAG layer can be compressed under low-magnitude loads (FIG. 16a-c). Unlike in the dry state, the hydrated unmineralised compartment does not fully regain its original thickness after the first application of compressive load (FIG. 16d), but instead drapes over the cross section of the mineralised compartment. After this initial compression, however, the unmineralised layer returns to its compressed thickness (FIG. 16d) after each subsequent application of compressive load. In FIG. 17, the ability of the unmineralised layer of a two-layer scaffold to adhere to the walls of a surgical defect encompassing the bone and cartilage interface in articular joints is illustrated by analogy. The glass slide in FIG. 17 is analogous to the wall of an osteochondral defect, and the ability of the unmineralised layer to adhere to this surface illustrates the capacity of these scaffolds to fill such defects to their periphery without the persistence of gaps between the unmineralised layer of the scaffold and the adjacent articular cartilage.

### Example III

### Three Layer Mineralised-Unmineralised Mineralised Scaffold

### Materials

Collagen (for mineralised slurry): Type I microfibrillar collagen from bovine tendon, Integra Life Sciences (Plainsboro, N.J., USA)
GAG (for mineralised slurry): Chondroitin-6-sulphate from shark cartilage, sodium salt, Sigma-Aldrich Inc (St. Louis, Mo., USA)
Calcium Sources: (i) Calcium hydroxide (Ca(OH)₂), Sigma-Aldrich Inc (St. Louis, Mo., USA); (ii) Calcium nitrate (Ca(NO₃)₂.4H₂O), Sigma-Aldrich Inc (St. Louis, Mo., USA)
Phosphorous Source: Orthophosphoric acid (H₃PO₄), BDH Laboratory Supplies (Poole, United Kingdom)
Collagen (for unmineralised collagen-GAG slurry): 85% Type I, 15% Type III Pepsin solubilised from porcine dermis, Japan Meat Packers (Osaka, Japan)
GAG (for unmineralised slurry): Chondroitin-6-sulphate from shark cartilage, sodium salt, Sigma-Aldrich Inc (St. Louis, Mo., USA)
Diluents for unmineralised Collagen and GAG: Glacial acetic acid (CH₃COOH), Fischer Scientific (Loughborough, UK)
Crosslinking Agents: Nordihydroguariaretic acid (NDGA), Sigma-Aldrich Inc (St. Louis, Mo., USA);
Sodium dihydrogen phosphate (NaH₂PO₄), BDH Laboratory Supplies (Poole, United Kingdom)
Sodium chloride (NaCl), Sigma-Aldrich Inc (St. Louis, Mo., USA)

### Step 0: Slurry Preparation

### Mineralised Slurry Preparation

3.8644 g collagen was dispersed in 171.4 mL of 0.1383M H₃PO₄ cooled in an ice bath by blending for 90 minutes at 15,000 rpm, using a homogeniser equipped with a 19 mm diameter stator to create a highly viscous collagen dispersion. In parallel, 0.3436 g chondroitin-6-sulphate (GAG) allowed to dissolve in 14.3 mL of 0.1383M H₃PO₄ at room temperature by shaking periodically to disperse dissolving GAG in order to produce a GAG solution. After 90 minutes, the 14.3 mL of GAG solution was added to the mixing collagen dispersion at a rate of approximately 0.5 mL/min, under continuous homogenisation at 15,000 rpm, and the resulting highly-viscous collagen/GAG dispersion blended for an additional 90 minutes. After 90 minutes of mixing, 1.804 g Ca(OH)₂ and 0.780 g Ca(NO₃)₂.4H₂O were added to the highly-viscous collagen/GAG dispersion over 30 minutes under constant blending at 15,000 rpm, creating a collagen/GAG/CaP slurry, the pH of which was approximately 4.0. The chilled slurry was then degassed in a vacuum flask over 25 hours at a pressure of 25 Pa, reblended using the homogenizer over 30 minutes, then degassed again for 48 hours.

### Unmineralised Slurry Preparation

1.9322 g of the Type I/III collagen was dispersed in 171.4 mL of 0.05M acetic acid cooled in an ice bath by blending for 90 minutes at 15,000 rpm, using a homogeniser equipped with a 19 mm diameter stator in order to create a highly viscous collagen dispersion. In parallel, 0.1718 g chondroitin-6-sulphate (GAG) was allowed to dissolve in 28.6 mL of 0.05M acetic acid at room temperature, by shaking periodically to disperse dissolving GAG in order to produce a GAG solution. After 90 minutes, the 14.3 mL of GAG solution was added to the mixing collagen dispersion at a rate of approximately 0.5 mL/min, under continuous homogenisation at 15,000 rpm, and the resulting highly-viscous collagen/GAG dispersion blended for an additional 90 minutes.

### Step I: Casting

3.5 mL of the mineralised collagen/GAG/CaP slurry was placed in the bottom portion of a combination polysulphone mould, the bottom portion of which measured 50 mm in length by 30 mm in width by 3 mm in depth. The slurry was smoothed to a flat surface using a razor blade. A middle collar, also made of polysulphone, and measuring 50 mm in length by 30 mm in width by 5 mm in depth, was attached to the bottom portion of the mould containing the smoothed, mineralised slurry. 7.5 mL of the unmineralised collagen/GAG slurry was placed, in an evenly distributed manner, on top of the smoothed, unmineralised layer and within the previously empty middle collar. An upper collar, also made of polysulphone and measuring 50 mm in length by 30 mm in width by 3 mm in depth, was attached to the middle portion of the mould above the smoothed, unmineralised slurry. 3.5 mL of the mineralised collagen/GAG/CaP slurry was placed, in an evenly distributed manner, on top of the smoothed, unmineralised layer and within the previously empty upper collar. All large bubbles were removed from the slurry using a hand pipettor

### Step II: Inter-Diffusion

The three-layer slurry was allowed to remain at room temperature and pressure for 20 minutes before being placed in the freeze dryer.

### Step III: Controlled Cooling

The mould and three-layer slurry were placed in a VirTis AdVantage freeze dryer (equipped with temperature-controlled, stainless steel shelves) and the shelf temperature of the freeze dryer ramped from 4° C. to −40° C. at a rate of approximately −2.4° C. per minute.

### Step IV: Annealing

The shelf temperature of the freeze dryer was maintained at −40° C. for 10 hours.

### Step V: Sublimation

While still at a shelf temperature of −40° C., a vacuum of below 25 Pa (approximately 200 mTorr) was applied to the chamber containing the mould and the (now frozen) three-layer slurry. The temperature of the chamber was then raised to 37° C., and sublimation allowed to continue for 36 hours. The vacuum was then removed, and the temperature returned to room temperature, leaving a three-layered scaffold 50 mm by 30 mm by 11 mm in size, comprised of an unmineralised middle layer 5 mm thick, surrounded by two mineralised layers 3 mm thick.

### Step VI: Crosslinking

The three-layer scaffold was hydrated in 0.1M NaH2PO4 and 0.15M NaCl in phosphate buffered saline (PBS; pH 7.0) for 30 minutes. NDGA was suspended in 1N NaOH and added to PBS to produce a 3 mg/mL solution of NGDA in PBS; scaffolds were then hydrated in this solution under agitation for 24 hours. The three-layer scaffold was removed from the NGDA-PBS solutions and rinsed with deionised water. The scaffolds were then freeze-dried to remove any residual water by controlled cooling from room temperature to −20° C. at a rate of approximately 2.4° C. per minute, followed by annealing at −20° C. for 5 hours, and finally sublimation at below 25 Pa at 37° C. for 24 hours, resulting in a dry, crosslinked scaffold. A subsequent treatment was then performed at a concentration of 0.1 mg/mL NDGA. The scaffolds were then washed in 70% ethanol for 6 hours and subsequently washed for 24 hours in PBS at room temperature. The scaffolds were then freeze dried for a second time to remove any residual water by controlled cooling from room temperature to −20° C. at a rate of approximately 2.4° C. per minute, followed by annealing at −20° C. for 5 hours, and finally sublimation at below 25 Pa at 37° C. for 24 hours.

The parameters in the Tables below are applicable singularly or in combination to any aspect of the present invention unless otherwise stated.

TABLE 1
Preferred Parameters for Casting

| Starting | Preferable | 0 to 37° | C. |
|---|---|---|---|
| Temperature for | More Preferable | 2 to 37° | C. |
| Controlled Cooling | Most Preferable | 4 to 37° | C. |
| Layer Thickness | Preferable | 0.1-500 | mm |
| | More Preferable | 0.5-20 | mm |
| | Most Preferable | 1.0-10 | mm |
| Slurry Viscosity | Preferable | 0.1-50 | Pa · s |
| | More Preferable | 0.1-10 | Pa · s |
| | Most Preferable | 0.5-5 | Pa · s |
| Thickness of Mould | Preferable | 1-50 | mm |
| Walls | More Preferable | 5-20 | mm |
| | Most Preferable | 5-15 | mm |
| Number of Layers | Preferable | 1-50 | |
| | More Preferable | 1-5 | |
| | Most Preferable | 1-3 | |

TABLE 2
Preferred Parameters for Inter-diffusion

| | | | |
|---|---|---|---|
| Time Allowed for | Preferable | 0-24 | hours |
| Inter-diffusion | More Preferable | 0-6 | hours |
| | Most Preferable | 0-2 | hours |
| Temperature | Preferable | 2-40° | C. |
| | More Preferable | 4-37° | C. |
| | Most Preferable | 20-37° | C. |
| Pressure | Preferable | 1-200 | kPa |
| | More Preferable | 50-150 | kPa |
| | Most Preferable | 50-101.325 | kPa |

TABLE 3
Preferred Parameters for Controlled Cooling

| | | | |
|---|---|---|---|
| Cooling Rate | Preferable | 0.02-10.0° | C./min |
| | More Preferable | 0.02-6.0° | C./min |
| | Most Preferable | 0.2-2.7° | C./min |
| Final Cooling | Preferable | −100 to 0° | C. |
| Temperature | More Preferable | −80 to −10° | C. |
| | Most Preferable | −40 to −20° | C. |

TABLE 4
Preferred Parameters for Annealing

| Annealing | Preferable | −100 to 0° | C. |
|---|---|---|---|
| Temperature | More Preferable | −80 to −10° | C. |
| | Most Preferable | −40 to −20° | C. |
| Annealing Time | Preferable | 0-48 | hours |
| | More Preferable | 2-12 | hours |
| | Most preferable | 8-10 | hours |

TABLE 5
Preferred Parameters for Sublimation

| | | | |
|---|---|---|---|
| Sublimation | Preferable | 0-0.08 | kPa |
| Pressure | More Preferable | 0.0025-0.08 | kPa |
| | Most Preferable | 0.0025-0.04 | kPa |
| Sublimation Time | Preferable | 0-120 | hours |
| | More Preferable | 12-72 | hours |
| | Most Preferable | 24-36 | hours |
| Sublimation | Preferable | −10-60° | C. |
| Temperature | More Preferable | 0-40° | C. |
| | Most Preferable | 20-37° | C. |

### REFERENCES

Gao J, Dennis J E, Solchaga L A, Awadallah A S, Goldberg V M, Caplan A I. 2001. Tissue-Engineered Fabrication of an Osteochondral Composite Graft Using Rat Bone Marrow-Derived Mesenchymal Stem Cells. Tissue Engineering 7:363-371.
Gao J, Dennis J E, Solchaga L A, Goldberg V M, Caplan A I. 2002. Repair of Osteochondral Defect with Tissue-Engineered Two-Phase Composite Material of Injectable Calcium Phosphate and Hyaluronan Sponge. Tissue Engineering 8:827-837.
Hung C T, Lima E G, Mauck R L, Taki E, LeRoux M A, Lu H H, Stark R G, Guo X E, Ateshian G A. 2003. Anatomically Shaped Osteochondral Constructs for Articular Cartilage Repair. Journal of Biomechanics 36:1853-1864.
Hunziker E B, Driesang I M K. 2003. Functional Barrier Principle for Growth-Factor-Based Articular Cartilage Repair. Osteoarthritis and Cartilage 11:320-327.
Niederauer G G, Slivka M A, Leatherbury N C, Korvick D L, H.H. H J, Ehler W C, Dunn C J, Kieswetter K. 2000. Evaluation of Multiphase Implants for Repair of Focal Osteochondral Defects in Goats. Biomaterials 21:2561-2574.
O'Brien F J, Harley B A, Yannas I V, Gibson L. 2004. Influence of Freezing Rate on Pore Structure in Freeze-Dried Collagen-GAG Scaffolds. Biomaterials 25:1077-1086.
O'Brien F J, Harley B A, Yannas I V, Gibson L J. 2005. The Effect of Pore Size and Structure on Cell Adhesion in Collagen-GAG Scaffolds. Biomaterials 26:433-441.
H M Loree, I V Yannas, B Mikic, A S Chang, S M Perutz, T V Norregaard, and C Kararup, ‘A freeze-drying process for fabrication of polymeric bridges for peripheral nerve regeneration’ Proc. 15th Annual Northeast Bioeng. Conf. P. 53-54, 1989.
Schaefer D, Martin I, Jundt G, Seidel J, Heberer M, Grodzinsky A, Bergin I, Vunjak-Novakovic G, Freed L E. 2002. Tissue-Engineered Composites for the Repair of Large Osteochondral Defects. Arthritis and Rheumatism 46:2524-2534.
Schaefer D, Martin I, Shastri P, Padera R F, Langer R, Freed L E, Vunjak-Novakovic G. 2000. In Vitro Generation of Osteochondral Composites. Biomaterials 21:2599-2606.
Sherwood J K, Riley S L, Palazzolo R, Brown S C, Monkhouse D C, Coates M, Griffith L G, Landeen L K, Ratcliffe A. 2002. A Three-Dimensional Osteochondral Composite Scaffold for Articular Cartilage Repair. Biomaterials 23:4739-4751.
Yannas I V, Lee E, Orgill D P, Skrabut E M, Murphy G F. 1989. Synthesis and Characterization of a Model Extracellular Matrix that Induces Partial Regeneration of Adult Mammalian Skin. Proceedings of the National Academy of Sciences of the United States of America 86:933-937.

The present invention finds application in a number of areas and the following are provided by way of example.

### Articular Cartilage Repair Product: Two-Layer Scaffold

Two layer scaffolds hold the potential to enhance the efficacy of existing first-line surgical procedures that recruit marrow-derived stem cells to the site of articular-cartilage injury. Delivered as, for example, a dry, 2 cm×2 cm×1 cm block of dry, vacuum-packed, gamma-sterilised material resembling styrofoam, these scaffolds can be cut using a scalpel or other tools, are easily inserted into the defect using simple thumb- or blunt-instrument pressure, and bond directly to the site without sutures or glue.

### Patellar Ligament Donor-Site Repair Product: Three Layer Scaffolds

Three-layer scaffolds hold the potential to enhance regeneration at patellar ligament (patella tendon) donor sites during anterior cruciate ligament (ACL) reconstruction, reducing frontal knee pain and reducing the risk of patellar ligament rupture and patellar fracture.

### Tendon Repair Product: Two-Layer Scaffolds

Two-layer scaffolds with extended unmineralised components hold the potential to improve the efficacy of tendon repair during rotator-cuff procedures and to address small-tendon applications for which no effective solution currently exists.

The present invention has been further studied on the basis of large-animal trials and a summary is presented below.

### Trial 1: Ovine Bone Defect Model

The present invention enables the production of layered tissue regeneration scaffolds whose structure and composition mimic bone on one side, unmineralised tissue (e.g. cartilage, ligament, tendon) on the other side, and a smooth, stable interface in between. The present invention furthermore offers the capacity to systematically alter the chemical composition of the mineral phase of the bony compartment of such implants.

Animal: skeletally mature Texcel Continental sheep (female).
Defect: 9 mm diameter by 9 mm deep cancellous bone defect on lateral femoral condyle.
Implantation Period: 6 weeks.
Experimental Groups Six implants of each experimental group implanted contralaterally with the same implant type in each side of the same animal.

### Control Groups:

Positive Control: four sites were filled with cancellous autograft harvested from the tibial tuberosity.
Negative Control: four sites were filled with control implants comprising implants containing no mineral phase at all (i.e. containing the organic constituents of the bony side of ChondroMimetic only).
Study Objective: to identify differences in the performance of four experimental implant groups differentiated by chemical composition and to identify the most desirable of these as the final composition for the bone compartment of ChondroMimetic.
Significant Findings: none of the three experimental groups invoked adverse immune responses of any kind; all three experimental groups plus the unmineralised negative control group supported bony in-growth via a cell-mediated direct substitution mechanism; no statistically significant differences between there three implant groups were observed; and bone formation observed in all three experimental groups was higher than that in the negative control group to a statistically significant level.
Implications for Implant Design: The direct substitution mechanism implied by this study suggests that the bone formation mechanism more closely resembles the templated bone formation that occurs at the growth plate in foetal and neonatal animals (including humans) than the typical apposition mechanism observed in traditional bone-graft substitutes. The presence of this substitution mechanism in the unmineralised control suggests that it is the organic constituent of the implants that imparts this character.

Pore size for the implants should be altered to account for this substitution mechanism by reducing the mean pore size of the bony compartment of the implants.

Lack of statistically significant differences in the bone formation behaviour of the three experimental groups suggests that processing parameters may be used to identify the most appropriate mineral composition of the implants.

### Trial 2: Caprine Osteochondral Defect Model

The objective of this study was to evaluate the performance of ChondroMimetic as a means of improving the results of a marrow stimulation technique (subchondral drilling).

Animal: skeletally mature Spanish-goats (female).
Defect: 4 mm diameter by 6 mm deep osteochondral defects (1 in trochlear groove; 1 on the lateral condyle).
Implantation Period: 16 weeks.
Experimental Groups Six implants of the ChondroMimetic working prototype.
Control Group: Six defects simulating traditional subchondral drilling (i.e. containing no implants).
Study Objective: to evaluate the performance of ChondroMimetic as an aid to marrow stimulation
Findings: feedback from surgeons about the handling characteristics of ChondroMimetic was, without exception, overwhelmingly positive.

### Annex 1

Text of description of PCT/GB04/004550, filed 28 Oct. 2004.

The present invention relates to the field of synthetic bone, dental materials and regeneration scaffolds for biomedical applications and, in particular, to synthetic bone, dental materials and regeneration scaffolds and their precursors comprising collagen, a calcium phosphate material and one or more glycosaminoglycans.

Natural bone is a biocomposite of collagen, non-collagenous organic phases including glycosaminoglycans, and calcium phosphate. Its complex hierarchical structure leads to exceptional mechanical properties including high stiffness, strength, and fracture toughness, which in turn enable bones to withstand the physiological stresses to which they are subjected on a daily basis. The challenge faced by researchers in the field is to make a synthetic material that has a composition and structure that will allow natural bone growth in and around the synthetic material in the human or animal body.

It has been observed that bone will bond directly to calcium phosphates in the human body (a property referred to as bioactivity) through a bone-like apatite layer formed in the body environment. Collagen and copolymers comprising collagen and other bioorganics such as glycosaminoglycans on the other hand, are known to be optimal substrates for the attachment and proliferation of numerous cell types, including those responsible for the production and maintenance of bone in the human body.

Hydroxyapatite is the calcium phosphate most commonly used as constituent in bone substitute materials. It is, however, a relatively insoluble material when compared to other forms of calcium phosphate materials such as brushite, tricalcium phosphate and octacalcium phosphate. The relatively low solubility of apatite can be a disadvantage when producing a biomaterial as the rate of resorption of the material in the body is particularly slow.

Calcium phosphates such as hydroxyapatite are mechanically stiff materials. However, they are relatively brittle when compared to natural bone. Collagen is a mechanically tough material, but has relatively low stiffness when compared to natural bone. Materials comprising copolymers of collagen and glycosaminoglycans are both tougher and stiffer than collagen alone, but still have relatively low stiffness when compared to natural bone.”

Previous attempts in the prior art of producing a synthetic bone-substitute material having improved mechanical toughness over hydroxyapatite and improved stiffness over collagen and copolymers of collagen and glycosaminoglycans include combining collagen and apatite by mechanical mixing. Such a mechanical method is described in EP-A-0164 484.

Later developments in the technology include producing a bone-replacement material comprising hydroxyapatite, collagen and chondroitin-4-sulphate by the mechanical mixing of these components. This is described in EP-A-0214070. This document further describes dehydrothermic crosslinking of the chondroitin-4-sulphate to the collagen. Materials comprising apatite, collagen and chondroitin-4-sulphate have been found to have good biocompatibility. The mechanical mixing of the apatite with the collagen, and optionally chondroitin-4-sulphate, essentially forms collagen/chondroitin-4-sulphate-coated particles of apatite. It has been found that such a material, although biocompatible, produces limited in-growth of natural bone when in the human or animal body and no remodeling of the calcium phosphate phase of the synthetic material.

The present invention seeks to address at least some of the problems associated with the prior art.

In a first aspect, the present invention provides a process for the production of a composite material comprising collagen, brushite and one or more glycosaminoglycans, said process comprising the steps of

providing an acidic aqueous solution comprising collagen, a calcium source and a phosphorous source and one or more glycosaminoglycans, and

precipitating the collagen, the brushite and the one or more glycosaminoglycans together from the aqueous solution to form a triple co-precipitate.

The term triple co-precipitate encompasses precipitation of the three compounds where the compounds have been precipitated at substantially the same time from the same solution/dispersion. It is to be distinguished from a material formed from the mechanical mixing of the components, particularly where these components have been precipitated separately, for instance in different solutions. The microstructure of a co-precipitate is substantially different from a material formed from the mechanical mixing of its components.

In the first aspect, the solution preferably has a pH of from 2.5 to 6.5, more preferably from 2.5 to 5.5. More preferably, the solution has a pH of from 3.0 to 4.5. Still more preferably, the solution has a pH of from 3.8 to 4.2. Most preferably, the solution has a pH of around 4.

The calcium source is preferably selected from one or more of calcium nitrate, calcium acetate, calcium chloride, calcium carbonate, calcium alkoxide, calcium hydroxide, calcium silicate, calcium sulphate, calcium gluconate and the calcium salt of heparin. A calcium salt of heparin may be derived from the porcine intestinal mucosa. Suitable calcium salts are commercially available from Sigma-Aldrich Inc.

The phosphorus source is preferably selected from one or more of ammonium-dihydrogen phosphate, diammonium hydrogen phosphate, phosphoric acid, disodium hydrogen orthophosphate 2-hydrate (Na₂HPO₄.2H₂O, sometimes termed GPR Sorensen's salt) and trimethyl phosphate, alkali metal salts (e.g Na or K) of phosphate, alkaline earth salts (e.g. Mg or Ca) of phosphate.

Glycosaminoglycans are a family of macromolecules containing long unbranched polysaccharides containing a repeating disaccharide unit. Preferably, the one or more glycosaminoglycans are selected from chondroitin sulphate, dermatin sulphate, heparin, heparin sulphate, keratin sulphate and hyaluronic acid. Chondroitin sulphate may be chondroitin-4-sulphate or chondroitin-6-sulphate, both of which are available from Sigma-Aldrich Inc. The chondroitin-6-sulphate may be derived from shark cartilage. Hyaluronic acid may be derived from human umbilical chord. Heparin may be derived from porcine intestinal mucosa.

Preferably, in the precipitation of the triple co-precipitate, the solution has a temperature of from 4.0 to 50° C. More preferably, the solution has a temperature of from 15 to 40° C. The solution may be at room temperature, that is from 20 to 30° C., with a temperature of from 20 to 27° C. being preferred. Most preferably, the temperature is around 25° C.

The concentration of calcium ions in the aqueous solution is typically from 0.00025 to 1 moldm⁻³ and preferably from 0.001 to 1 moldm⁻³. Where the process includes the additional further steps of filtration and/or low temperature drying, the concentration of calcium ions in the aqueous solution is more preferably from 0.05 to 0.5 moldm⁻³ (for example from 0.08 to 0.25 moldm⁻³) and most preferably from 0.1 to 0.5 moldm⁻³. Where the process includes the additional further steps of freeze drying and optionally injection moulding, the concentration of calcium ions in the aqueous solution is more preferably from 0.01 to 0.3 moldm⁻³ and most preferably from 0.05 to 0.18 moldm⁻³.

Preferably, the solution comprises phosphate ions and the concentration of phosphate ions in solution is typically from 0.00025 to 1 moldm⁻³ and preferably from 0.001 to 1 M. Where the process includes the additional further steps of filtration and/or low temperature drying, the concentration of phosphate ions in solution is more preferably 0.05 to 0.5 moldm⁻³, still more preferably 0.1 to 0.5 M, for example 0.1 to 0.35 moldm⁻³. Where the process includes the additional further steps of freeze drying and optionally injection moulding, the concentration of phosphate ions in solution is more preferably from 0.01 to 0.3 moldm⁻³, still more preferably 0.05 to 0.18 M.

Preferably, the ratio of collagen to the total amount of one or more glycosaminoglycans in the solution prior to precipitation is from 8:1 to 30:1 by weight. More preferably, the ratio of collagen to the total amount of one or more glycosaminoglycans is from 10:1 to 12:1, and most preferably the ratio is from 11:1 to 23:2.

Preferably, the ratio of collagen to brushite in the triple co-precipitate is from 10:1 to 1:100 by weight, more preferably from 5:1 to 1:20, still more preferably from 3:2 to 1:10, most preferably from 3:2 to 1:4.

The concentration of collagen in the solution prior to precipitation is typically from 1 to 20 g/L, more preferably from 1 to 10 g/L. Where the process includes the steps of filtration and/or low temperature drying, the concentration of collagen in the solution is more preferably from 1 to 10 g/L, still more preferably from 1.5 to 2.5 g/L, and most preferably 1.5 to 2.0 g/L. Where the process includes freeze drying and optionally injection moulding, the concentration of collagen in the solution prior to precipitation is preferably from 5 to 20 g/L, more preferably from 5 to 12 g/L, and most preferably from 9 to 10.5 g/L.

The total concentration of the one or more glycosaminoglycans in the solution prior to precipitation is typically from 0.01 to 1.5 g/L, more preferably from 0.01 to 1 g/L. Where the process includes the additional further steps of filtration and/or low temperature drying, the total concentration of the one or more glycosaminoglycans in the solution is more preferably from 0.03 to 1.25 g/L, still more preferably from 0.125 to 0.25 g/L, and most preferably from 0.13 to 0.182 g/L. Where the process includes the additional further steps of freeze drying and optionally injection moulding, the total concentration of the one or more glycosaminoglycans in the solution is more preferably from 0.15 to 1.5 g/L, still more preferably from 0.41 to 1.2 g/L, and most preferably from 0.78 to 0.96 g/L.

Preferably the solution comprises calcium ions and the ratio of collagen to the calcium ions is typically from 1:40 to 500:1 by weight. Where the process includes the additional further steps of filtration and/or low temperature drying, the ratio of collagen to the calcium ions is more preferably from 1:40 to 250:1, still more preferably 1:13 to 5:4, and most preferably 1:13 to 1:2. Where the process includes the additional further steps of freeze drying and optionally injection moulding, the ratio of collagen to the calcium ions is more preferably from 1:8 to 500:1, still more preferably 5:12 to 30:1, and most preferably 5:5 to 5:1.

Precipitation may be effected by combining the collagen, the calcium source, the phosphorous source and one or more glycosaminoglycans in an acidic aqueous solution and either allowing the solution to stand until precipitation occurs, agitating the solution, titration using basic titrants such as ammonia, addition of a nucleating agent such as pre-fabricated brushite, varying the rate of addition of the calcium source, and any combination of these techniques.

In a second aspect, the present invention provides a process for the production of a composite biomaterial comprising collagen, octacalcium phosphate and one or more glycosaminoglycans, said process comprising the steps of

providing a composite material comprising collagen, brushite and one or more glycosaminoglycans, and

converting at least some of the brushite in the composite material to octacalcium phosphate by hydrolysation.

The term biomaterial encompasses a material that is biocompatible with a human or animal body.

In the second aspect, the composite material preferably comprises or consists essentially of a triple co-precipitate comprising collagen, brushite and one or more glycosaminoglycans. The triple co-precipitate may be formed by a process as herein described in relation to the first aspect of the present invention.

Preferably, the step of hydrolysation (hydrolysis) of brushite to octacalcium phosphate comprises contacting the triple co-precipitate with an aqueous solution, said aqueous solution being at or above the pH at which octacalcium phosphate becomes thermodynamically more stable than brushite. Preferably, this aqueous solution has a pH of from 6 to 8. More preferably, this aqueous solution has a pH of from 6.3 to 7. Most preferably, this aqueous solution has pH of about 6.65. The aqueous solution may comprise, for example, deionised water whose pH is controlled with a titrant, a buffer solution, a solution saturated with respect to another calcium-containing compound and/or phosphorus-containing compound. A preferred aqueous solution comprises acetic acid titrated to the desired pH using ammonia.

Preferably, the step of hydrolysation of brushite to octacalcium phosphate is preformed at a temperature of from 20 to 50° C., more preferably from 30 to 40° C., still more preferably from 36 to 38° C., most preferably around 37° C.

Preferably, the step of hydrolysation of brushite to octacalcium phosphate is preformed for a time of from 12 to 144 hours, more preferably from 18 to 72 hours, most preferably from 24 to 48 hours.

In a third aspect, the present invention provides a process for the production of a composite biomaterial comprising collagen, apatite and one or more glycosaminoglycans, said process comprising the steps of

providing a composite material comprising collagen, brushite and one or more glycosaminoglycans, and

converting at least some of the brushite in the composite material to apatite by hydrolysation.

Apatite is a class of minerals comprising calcium and phosphate and has the general formula: Ca₅(PO₄)₃(X), wherein X may be an ion that is typically OH⁻, F⁻ and Cl⁻, as well as other ions known to those skilled in the art. Apatite also includes substituted apatites such as silicon-substituted apatites. Apatite includes hydroxyapatite, which is a specific example of an apatite. The hydroxyapatite may also be substituted with silicon.

In the third aspect, the composite material preferably comprises or consists essentially of a triple co-precipitate comprising collagen, brushite and one or more glycosaminoglycans. The triple co-precipitate may be formed according to the process as herein described in relation to the first aspect of the present invention.

Preferably, the step of hydrolysation (hydrolysis) of brushite to apatite comprises contacting the triple co-precipitate with an aqueous solution, said aqueous solution being at or above the pH at which apatite becomes thermodynamically more stable than brushite. Preferably, for the conversion of brushite to apatite, the aqueous solution has a pH of from 6.65 to 9, more preferably from 7 to 8.5, still more preferably from 7.2 to 8.5. The aqueous solution may comprise, for example, deionised water whose pH is controlled with a titrant, a buffer solution, a solution saturated with respect to another calcium-containing compound and/or phosphorus-containing compound.

Preferably, the step of hydrolysation of brushite to apatite is performed at a temperature of 20 to 50° C., more preferably from 30 to 40° C., still more preferably from 36 to 38° C., most preferably around 37° C.

Preferably, the step of hydrolysation of brushite to apatite is performed for a time of from 12 to 288 hours, more preferably from 18 to 72 hours, most preferably from 24 to 48 hours.

Methods of increasing the rate of conversion of brushite to octacalcium phosphate and/or apatite include (i) increasing the temperature, (ii) the brushite concentration in solution, and/or (iii) the agitation speed.

It may be desirable to produce a biomaterial according to the present invention comprising both apatite and octacalcium phosphate. The processes of the second and third aspects of the present invention may be combined to produce a material comprising both octacalcium phosphate and apatite. The brushite in the triple co-precipitate may first be converted to octacalcium phosphate and then the octacalcium phosphate may be partially converted to apatite. Total, or near total (i.e. at least 98%), conversion of brushite or octacalcium phosphate to apatite typically occurs by hydrolysation at a pH of 8.0 or more for a period of about 12 hours. Partial conversion of the brushite and/or apatite in the material may therefore be effected by hydrolysation for a period of less than 12 hours.

Preferably, the step of hydrolysation of octacalcium phosphate to apatite is carried out at a pH of from 6.65 to 10, more preferably from 7.2 to 10, still more preferably from 8 to 9.

Preferably, the step of hydrolysation of octacalcium phosphate to apatite is performed at a temperature of from 20 to 50° C., more preferably from 30 to 40° C., still more preferably from 36 to 38° C., most preferably around 37° C.

Preferably, the step of hydrolysation of octacalcium phosphate to apatite is performed for a time of from 2 to 144 hours, more preferably from 12 to 96 hours, most preferably from 24 to 72 hours.

In the second and third aspects of the present invention, the conversion of brushite to octacalcium phosphate and/or apatite is preferably conducted at a temperature of from 30 to 40 degrees centigrade. More preferably, the conversion is conducted at a temperature of from 36 to 38 degrees centigrade. Most preferably, the conversion is conducted at a temperature of about 37 degrees centigrade.

Preferably, the processes of the present invention further comprise the step of crosslinking the one or more glycosaminoglycans and the collagen in the triple co-precipitate. By triple co-precipitate this includes the triple co-precipitate comprising collagen, brushite and one or more glycosaminoglycans and derivatives of the co-precipitate. Derivatives include the co-precipitate wherein at least some of the brushite has been converted to octacalcium phosphate and/or apatite, and the co-precipitate that has been shaped or moulded, or subjected to any further chemical or mechanical processing. Crosslinking may be achieved using any of the conventional techniques.

Preferably, at least some of the brushite is converted to octacalcium phosphate and/or apatite, the glycosaminoglycan and collagen are crosslinked prior to the conversion of the brushite to octacalcium phosphate and/or apatite. This crosslinking may be effected by subjecting the triple co-precipitate to one or more of gamma radiation, ultraviolet radiation, a dehyrdothermal treatment, non-enzymatic glycation with a simple sugar such as glucose, mannose, ribose and sucrose, contacting the triple co-precipitate with one or more of glutaraldehyde, ethyl dimethylaminopropyl carbodiimide and/or nor-dihydroguariaretic acid, or any combination of these methods. These methods are conventional in the art.

Preferably, if at least some of the brushite is converted to octacalcium phosphate and/or apatite, the glycosaminoglycan and collagen are crosslinked subsequent to the conversion of the brushite to octacalcium phosphate and/or apatite. The crosslinking subsequent to the conversion of the brushite to apatite/octacalcium phosphate may be effected by one or more of the methods mentioned above or a dehydrothermal treatment, or any combination of these methods. A dehydrothermal treatment includes subjecting a substrate to a low pressure atmosphere at a raised temperature. The temperature in the dehydrothermal treatment may be of from 95° C. to 135° C. The temperature may preferably be of from 100° C. to 110° C., and most preferably of from 105° C. to 110° C., if completion of the dehydrothermal treatment is desired in typically 18 to 36 hours. The temperature may preferably be of from 120° C. to 135° C., and most preferably of from 125° C. to 135° C., if completion of the dehydrothermal treatment is desired in typically 4 to 8 hours.

Preferably, the collagen and the glycosaminoglycan are crosslinked both prior to and subsequent to conversion of the brushite to octacalcium phosphate and/or apatite.

The processes of the present invention may comprise the step of shaping the composite biomaterial into a structure suitable for use as a bone or dental substitute. Such a step may occur after formation of the triple co-precipitate, but prior to any conversion of the brushite or crosslinking of the collagen and glycosaminoglycan that may occur.

Alternatively, the step of shaping the biomaterial may occur subsequent to either the conversion of the brushite to apatite and/or octacalcium phosphate or crosslinking of the collagen and the glycosaminoglycan.

Preferably, the composite material is shaped using a technique selected from (i) filtration and/or low temperature drying, (ii) freeze drying, (iii) injection moulding and (iv) cold pressing. Filtration and/or low temperature drying, wherein the temperature is from 15° C. to 40° C., most preferably of from 35° C. to 40° C., typically results in a dense granular form of material. Freeze drying typically results in an open porous form. Injection moulding results in a wide variety of shapes/morphologies of a material depending on the shape of the dye used. Cold pressing typically results in a dense pellet form.

The present invention further provides a precursor material suitable for transforming into a synthetic biomaterial, said precursor material comprising a composite material comprising collagen, brushite and one or more glycosaminoglycans. Preferably, the composite material comprises or consists essentially of a triple co-precipitate comprising collagen, brushite and one or more glycosaminoglycans. The triple co-precipitate may be produced according to the process of the first aspect of the present invention.

The present invention also provides a composite biomaterial comprising collagen, brushite and one or more glycosaminoglycans, which biomaterial is obtainable by a process according to the present invention as herein described.

The present invention also provides a composite biomaterial comprising collagen, octacalcium phosphate and one or more glycosaminoglycans, which biomaterial is obtainable by a process according to the second aspect of the present invention.

The present invention also provides a composite biomaterial comprising collagen, apatite and one or more glycosaminoglycans, which biomaterial is obtainable by a process according to the third aspect of the present invention.

The present invention also provides a composite biomaterial comprising a triple co-precipitate of collagen, glycosaminoglycan and brushite.

The present invention also provides a biomaterial comprising particles of one or more calcium phosphate materials, collagen and one or more glycosaminoglycans, wherein said collagen and said one or more glycosaminoglycans are crosslinked and form a matrix, said particles of calcium phosphate material are dispersed in said matrix, and said calcium phosphate material is selected from one or more of brushite, octacalcium phosphate and/or apatite.

The following description relates to all aspects of the composite biomaterial according to the present invention unless otherwise stated.

The collagen and the one or more glycosaminoglycans have preferably been crosslinked.

The collagen is preferably present in the material in an amount of from 5 to 90 (dry) wt %, more preferably from 15 to 60 (dry) wt %, %, more preferably from 20 to 40 (dry) wt %.

Preferably, the one or more glycosaminoglycans are present in the material in an amount of from 0.01 to 12 (dry) wt %, more preferably from 1 to 5.5 (dry) wt %, most preferably from 1.8 to 2.3 (dry) wt %.

Preferably, if the material comprises brushite, the ratio of collagen to brushite is 10:1 to 1:100 by weight (dry), more preferably 5:1 to 1:20 by weight (dry), most preferably 3:2 to 1:10 by weight (dry), for example 3:2 to 1:4 by weight (dry).

Preferably if the material comprises octacalcium phosphate, the ratio of collagen to octacalcium phosphate is 10:1 to 1:100 by weight (dry), more preferably 5:1 to 1:20 by weight (dry), most preferably 3:2 to 1:10 by weight (dry).

Preferably, the ratio of collagen to the total amount of one or more glycosaminoglycans is from 8:1 to 30:1 by weight (dry), more preferably from 10:1 to 30:1 by weight (dry), still more preferably 10:1 to 12:1 by weight (dry), and most preferably 11:1 to 23:2 by weight (dry).

The composite biomaterial according to the present invention may be used as a substitute bone or dental material.

The present invention also provides a synthetic bone material, bone implant, bone graft, bone substitute, bone scaffold, filler, coating or cement comprising a composite biomaterial of the present invention. The term coating includes any coating comprising the biomaterial or precursor of the present invention. The coating may be applied to the external or internal surfaces of prosthetic members, bones, or any substrate intended for use in the human or animal body, which includes particulate materials. The composition of the present invention may be used for both in-vivo and ex-vivo repair of both mineralized biological material, including but not limited to bone and dental materials. The biomaterials of the present invention may be used in the growth of allografts and autografts.

The biomaterial according to the present invention comprising octacalcium phosphate may by free or essentially free of any of the precursor brushite phase. This biomaterial may comprise less than 2% by weight of brushite in total amount of calcium phosphate materials in the biomaterial.

The calcium phosphate material may comprise or consist essentially of phase pure octacalcium phosphate or apatite. By phase pure, this means preferably containing at least 98%, more preferably at least 99%, and most preferably, at least 99.5% of the desired phase (as measured by x-ray diffraction). Alternatively, the biomaterial may comprise a mixture of octacalcium phosphate and apatite, depending on the desired properties of the biomaterial.

The material of the present invention comprising brushite may be used either as a precursor material for making a biomaterial, or may be suitable in itself for use as a biomaterial.

The processes according to the present invention may be preformed using the following sequential method, which may be applied in whole or in part, to produce biocomposites of collagen, one or more glycosaminoglycan and one or more calcium phosphate constituents. The following description is provided by way of example and is applicable to any aspect of the processes according to the present invention.

### I: Triple Co-precipitation of Collagen, GAG, and the Calcium Phosphate Brushite at Acidic pH

This step is performed to initiate simultaneous formation, via precipitation from solution, of the three (or more) constituents of the composite, and to control the ratio of the three (or more) respective phases. Control of the compositional properties of the composite (and in particular the collagen:GAG:CaP ratio) may be achieved by varying one or more of the pH, temperature, ageing time, calcium ion concentration, phosphorous ion concentration, collagen concentration and GAG concentration. The pH may be maintained constant (using, for example, buffers, pH-stat titration or other methods) or be allowed to vary. The possible secondary (contaminant) phases include other acidic calcium phosphates (e.g. monetite, calcium hydrogen phosphate) and complexes including by-products of titration and reactant addition (e.g. ammonium phosphate, ammonium nitrate). Additives to aid crosslinking (e.g. glucose, ribose) or to enhance in-vivo response (e.g. growth factors, gene transcription factors, silicon, natriuretic peptides) may also be added during this step.

### II: Net Shape Formation

This step may be performed to produce the desired architecture of the final composite form, with particular emphasis on control of pore architecture. Examples of techniques include filtration and low-temperature drying (resulting in a dense granular form), freeze drying (resulting in an open porous form), injection moulding (resulting in a wide range of shapes depending on the type of dye) and cold pressing (resulting in a dense pellet form).

### III: Primary Crosslinking

This step may be performed to preferably ensure that, when placed in a solution of elevated pH, the GAG content of the composite does not elude rapidly, and, furthermore, to enhance the mechanical and degradation properties of the composite. Examples of techniques include low-temperature physical techniques (e.g. gamma irradiation, ultraviolet radiation, dehydrothermal treatment), chemical techniques (e.g. non-enzymatic glycation with a simple sugar, glutaraldehyde, ethyl dimethylaminopropyl carbodiimide, nordihydroguariaretic acid), or combination methods (e.g. simultaneous non-enzymatic glycation and gamma-irradiation). In the event that conversion to octacalcium phosphate (i.e. as in step IV) is desirable, primary crosslinking is advantageously performed at a temperature below about 37° C. to prevent conversion of the brushite phase to its dehydrated form, monetite, which is a calcium phosphate that does not readily hydrolyse to octacalcium phosphate.

### IV: Hydrolysis

This step may be performed to partially or fully hydrolyse the CaP phase from brushite (phase with high solubility at physiological pH) to octacalcium phosphate and/or apatite (phases with lower solubility at physiological pH), and to substantially remove any soluble contaminant phases (e.g. ammonium nitrate, calcium hydrogen phosphate). In the case of hydrolysis to OCP, the selected pH is advantageously maintained constant at about 6.65 (using a buffer, pH stat, or other method), and the temperature at about 37° C. for around 24-48 hours. As was the case in Step I, additives to aid in crosslinking (e.g. glucose, ribose) or to enhance in-vivo response (e.g. growth factors, gene transcription factors, silicon, natriuretic peptides) may also be added during the hydrolysis step (Step IV).

### V: Secondary Crosslinking

This step may be performed to further tailor the mechanical and degradation properties of the composite. Any or all of the crosslinking procedures listed in Step III above may be used to effect secondary crosslinking.

The following Examples and the accompanying Figures are provided to further assist in the understanding the present invention. The Examples and Figures are not to be considered limiting to the scope of the invention. Any feature described in the Examples or Figures is applicable to any aspect of the foregoing description.

### Example 1

Example 1 is an example of the synthesis method described above, executed via application of steps I through III only. Triple co-precipitation is carried out at room temperature (20-25° C.), at a pH of about 3.2 (maintained by titration with ammonia). In this example, co-precipitates are dried at 37° C. and crosslinked via a dehydrothermal treatment. Neither hydrolytic conversion of the CaP nor secondary crosslinking is performed in this example.

### Materials

Collagen: Reconstituted, pepsin-extracted porcine dermal collagen (atelocollagen); 85% Type I, 15% Type III; Japan Meat Packers (Osaka, Japan)
GAG: Chondroitin-6-sulphate from shark cartilage; sodium salt; Sigma-Aldrich Inc (St. Louis, Mo., USA)
Calcium Sources: (i) Calcium hydroxide; Ca(OH)₂ Sigma-Aldrich Inc (St. Louis, Mo., USA), (ii) Calcium nitrate; Ca(NO₃)₂.4H₂O; Sigma-Aldrich Inc (St. Louis, Mo., USA)
Phosphorous Source: Orthophosphoric acid; H₃PO₄; BDH Laboratory Supplies (Poole, United Kingdom)

### Titrant: Ammonia; NH₃; BDH Laboratory Supplies (Poole, United Kingdom)

### Procedure

### Step I

### Solution A:

Ca(OH)₂ is dissolved in 0.48M H₃PO₄ to a concentration of 0.12M at room temperature, and the resulting solution titrated to pH=3.2 using ammonia.

### Suspension B:

Chondroitin-6-sulphate is dissolved in dionised water to a concentration of 3.2 g/L. Under constant stirring, Ca(NO₃)₂.4H₂O and Ca(OH)₂ is then added to the chondroitin-sulphate solution at a nitrate:hydroxide molar ratio of 1.5, to produce a suspension with a total calcium concentration of 2.4M.

0.144 g collagen is added to 20 mL of Solution A, and blended using a homogeniser until dissolved. 4 mL of Suspension B is then added to Solution A under constant stirring.

Stirring is continued for 60 minutes, and pH monitored to ensure that it remains in the range 3.15<pH<3.30. The resulting slurry is then allowed to age for 24 hours at room temperature.

### Step II

The slurry is allowed to dry at 37° C. in air for 5 days, and the remaining triple co-precipitate rinsed with deionised water, and subsequently dried again at 37° C. for an additional 24 hours.

The x-ray diffraction pattern of the resultant triple coprecipitate is shown in FIG. 1 (Cu-K(alpha) radiation) and an SEM image is shown in FIG. 2.

### Step III

Triple co-precipitates are crosslinked via dehydrothermal treatment (DHT) at 105° C., under a vacuum of 50 mTorr, for 48 hours. A TEM image of the triple co-precipitate following DHT is shown in FIG. 3. FIG. 4 shows the x-ray diffraction pattern of the triple co-precipitate following DHT and indicates that the brushite phase has converted to its dehydrated form monetite.

### Example 2

Example 2 is an example of the synthesis method described above, executed via application of steps I through IV only. Triple co-precipitation is carried out at room temperature, and a pH of 4.0. In this example, pH control is effected by careful control of the calcium hydroxide and calcium nitrate concentrations—an approach that also enables control of the mass ratio of brushite to collagen plus GAG in the triple coprecipitate. The resulting triple co-precipitates are then frozen to −20° C., placed under vacuum and then heated to induce sublimation of unbound water (i.e. ice). Primary crosslinking is performed using a 1-ethyl 3-(3-dimethyl aminopropyl) carbodiimide treatment. The resulting dried triple coprecipitate is then converted to octacalcium phosphate via hydrolysis at a pH of 6.67 at about 37° C. In this example, secondary crosslinking is not performed.

### Materials

Type I: Acid solubilised from bovine tendon Integra Life Sciences Plainsboro, N.J., USA
GAG: Chondroitin-6-sulphate from shark cartilage; sodium salt; Sigma-Aldrich Inc (St. Louis, Mo., USA)
Calcium Sources: (i) Calcium hydroxide; Ca(OH)₂ Sigma-Aldrich Inc (St. Louis, Mo., USA), and (ii) Calcium nitrate; Ca(NO₃)₂.4H₂O; Sigma-Aldrich Inc (St. Louis, Mo., USA)
Phosphorous Source: Orthophosphoric acid; H₃PO₄; BDH Laboratory Supplies (Poole, United Kingdom)

### Titrant: None

Crosslinking agents: (i) 1-Ethyl-3-(3-Dimethylaminopropyl) Carbodiimide (=EDAC); Sigma-Aldrich Inc (St. Louis, Mo., USA), and (ii) N-Hydroxysuccinimide (=NHS); Sigma-Aldrich Inc (St. Louis, Mo., USA)

### Procedure

### Step I

A target mass ratio of brushite to collagen plus glycosaminoglycan of 1:1 is selected.

The concentration of collagen plus GAG in a total reaction volume of 200 mL is set at 21 mg/mL.

Using an empirical, 3-dimentional map of pH variation (produced at a constant [Ca²⁺] to [P] reactant ion ratio of 1.0) with differing (i) ionic concentrations (i.e. [Ca²⁺]=[H₃PO₄]) and (ii) ratios of calcium nitrate:calcium hydroxide, a locus of points over which pH remained constant at 4.0 is identified. This is shown in FIG. 5 (sets of combinations of ionic concentration and calcium nitrate:calcium hydroxide ratio for maintaining pH=4.0).

Superimposing this locus of points onto a map of brushite mass yield with identical axes, and identification of its intersection with the 21 mg/mL contour allows the set of reactant concentrations for which a triple coprecipitate slurry containing a 1:1 mass ratio of calcium phosphate (21 mg/mL) to collagen plus GAG (21 mg/mL) can be produced at pH 4.0 ([Ca²⁺]=[H₃PO₄]=0.1383M; Ca(NO₃).4H₂O: Ca(OH)₂=0.1356) See FIG. 6: identification of conditions for pH 4.0 synthesis of a triple coprecipitate slurry containing a 1:1 mass ratio of calcium phosphate to collagen plus GAG.

3.8644 g collagen is dispersed in 171.4 mL of 0.1383M H₃PO₄ cooled in an ice bath, by blending over 90 minutes at 15,000 rpm, using a homogeniser equipped with a stator 19 mm in diameter, to create a highly viscous collagen dispersion.

0.3436 g chondroitin-6-sulphate (GAG) is allowed to dissolve in 14.3 mL of 0.1383M at room temperature, by shaking periodically to disperse dissolving GAG, producing a GAG solution.

After 90 minutes, the 14.3 mL of GAG solution is added to the mixing collagen dispersion at a rate of approximately 0.5 mL/min, under continuous homogenisation at 15,000 rpm, and the resulting highly-viscous collagen/GAG dispersion blended for a total of 90 minutes

After 90 minutes of mixing, 1.804 g Ca(OH)₂ and 0.780 g Ca(NO₃)₂.4H₂O are added to the highly-viscous collagen/GAG dispersion over 30 minutes under constant blending at 15,000 rpm, creating a collagen/GAG/CaP triple coprecipitate slurry, after which time an additional 14.3 mL of 0.1383M H₃PO₄ is blended into the slurry

The pH of the triple coprecipitate slurry is approximately 4.0

The triple coprecipitate slurry is allowed to remain at 25° C. for a period of 48 hours.

### Step II

The triple coprecipitate slurry is placed in a freezer at −20° C. and allowed to solidify overnight.

The frozen slurry is then removed from the freezer, placed in a vacuum of approximately 80 mTorr, and the temperature allowed to rise to room temperature, thus inducing sublimation of ice from the slurry, which is allowed to proceed over 48 hours.

The x-ray diffraction pattern of the collagen/GAG/brushite triple co-precipitate following removal of unbound water (Cu-K (alpha) radiation) is shown in FIG. 7, and an SEM image of the surface of a co-precipitate is shown in FIG. 8 (secondary (SE) and backscattered electron (BSE) images of surface of triple co-precipitate with CaP: collagen+GAG=1:1).

### Step III

After complete removal of unbound water, 1.25 g of the resulting dry triple coprecipitate is hydrated in 40 mL deionised water for 20 minutes.

20 mL of a solution of 0.035M EDAC and 0.014M NHS is added to the container containing the triple coprecipitates and deionised water, and the triple coprecipitates allowed to crosslink for 2 hours at room temperature under gentle agitation.

The EDAC solution is removed, and the triple coprecipitates rinsed with phosphate buffer solution (PBS) and allowed to incubate at 37° C. for 2 hours in fresh PBS under mild agitation.

After two hours in PBS, the triple coprecipitates are rinsed with deionised water, and allowed to incubate for two 10-minute intervals at 37° C. under mild agitation.

The triple coprecipitates are then dried at 37° C. for 72 hours. FIG. 9 shows an x-ray diffraction pattern of the collagen/GAG/brushite triple coprecipitate following EDAC crosslinking (Cu-K (alpha)-radiation).

### Step IV

Crosslinked triple coprecipitate granules are placed in 50 mL deionised water at 37° C., and the pH of the solution adjusted to 6.67 using ammonia.

Temperature and pH are maintained constant for 48 hours, after which time the co-precipitates are filtered, rinsed in deionised water, and dried at 37° C. in air.

An x-ray diffraction pattern of the coprecipitates following conversion to OCP is shown in FIG. 10 (EDAC-crosslinked collagen/GAG/CaP triple co-precipitate following conversion at 37° C. to OCP over 72 hours at pH 6.67, to form a collagen/GAG/OCP biocomposite, Cu-K (alpha) radiation).

### Example 3

Example 3 is an example of the synthesis method described above, executed via application of steps I through V inclusive. Triple co-precipitation is carried out at room temperature, and a pH of about 4.5. As in example 2, pH control is effected by careful control of the calcium hydroxide and calcium nitrate concentrations, without the use of titrants. The resulting co-precipitates are then frozen to −20° C., placed under vacuum and then heated to induce sublimation of unbound water (i.e. ice). Primary crosslinking is performed using a 1-ethyl 3-(3-dimethyl aminopropyl) carbodiimide treatment. The resulting dried coprecipitate is then converted to apatite at pH 8.50, at 37° C. Secondary crosslinking performed using gamma irradiation.

### Materials

Type I: Acid solubilised from bovine tendon Integra Life Sciences Plainsboro, N.J., USA
GAG: Chondroitin-6-sulphate from shark cartilage; sodium salt; Sigma-Aldrich Inc (St. Louis, Mo., USA)
Calcium Sources: (i) Calcium hydroxide; Ca(OH)₂ Sigma-Aldrich Inc (St. Louis, Mo., USA), and (ii) Calcium nitrate; Ca(NO₃)₂.4H₂O; Sigma-Aldrich Inc (St. Louis, Mo., USA)
Phosphorous Source: Orthophosphoric acid; H₃PO₄; BDH Laboratory Supplies (Poole, United Kingdom)

### Titrant: None

Crosslinking agents: (i) 1-Ethyl-3-(3-Dimethylaminopropyl) Carbodiimide (=EDAC); Sigma-Aldrich Inc (St. Louis, Mo., USA) and (ii) N-Hydroxysuccinimide (=NHS); Sigma-Aldrich Inc (St. Louis, Mo., USA)

### Procedure

### Step I

A target mass ratio of brushite to collagen plus glycosaminoglycan of 3:1 is selected.

The concentration of collagen plus GAG in a total reaction volume of 200 mL is set at 10 mg/mL.

Using an empirical, 3-dimentional map of pH variation (at a constant [Ca²⁺] to [P] reactant ion ratio of 1.0) with differing i) ionic concentrations (i.e. [Ca²⁺]=[H₃PO₄]) and ii) ratios of calcium nitrate: calcium hydroxide, a locus of points over which pH remained constant at 4.5 is identified. This is shown in FIG. 11 (set of combinations of ionic concentration and calcium nitrate:calcium hydroxide ratio for maintaining pH=4.5).

Superimposing this locus of points onto a map of brushite mass yield (with identical axes), and identification of its intersection with the 30 mg/mL (i.e. 3 times the concentration of collagen plus GAG) contour allows the set of reactant concentrations for which a triple coprecipitate slurry containing a 3:1 mass ratio of calcium phosphate (30 mg/mL) to collagen plus GAG (10 mg/mL) can be produced at a pH of 4.5 ([Ca²⁺]=[H₃PO₄]=0.1768M; Ca(NO₃).4H₂O: Ca(OH)₂=0.049). This is show in FIG. 12: identification of conditions for pH 4.5 synthesis of a triple coprecipitate slurry containing a 3:1 mass ratio of calcium phosphate to collagen plus GAG.

1.837 g collagen is dispersed in 171.4 mL of 0.1768M H₃PO₄ cooled in an ice bath, by blending over 90 minutes at 15,000 rpm, using a homogeniser equipped with a stator 19 mm in diameter, to create a collagen dispersion.

0.163 g chondroitin-6-sulphate (GAG) is allowed to dissolve in 14.3 mL of 0.1768M at room temperature, by shaking periodically to disperse dissolving GAG, to produce a GAG solution.

After 90 minutes, the 14.3 mL of GAG solution is added to the mixing collagen dispersion at a rate of approximately 0.5 mL/min, under continuous homogenisation at 15,000 rpm, and the resulting collagen/GAG dispersion blended for a total of 90 minutes.

After 90 minutes of mixing, 2.498 g Ca(OH)₂ and 0.380 g Ca(NO₃)₂.4H₂O are added to the collagen/GAG dispersion over 30 minutes under constant blending at 15,000 rpm, creating a collagen/GAG/CaP triple coprecipitate slurry, after which time an additional 14.3 mL of 0.1768M H₃PO₄ were added to the mixing slurry.

The pH of the triple coprecipitate slurry is approximately 4.5.

The triple coprecipitate slurry is allowed to remain at 25° C. for a period of 48 hours.

### Step II

The triple coprecipitate slurry is placed in a freezer at −20° C. and allowed to freeze overnight.

The frozen slurry is then removed from the freezer, placed in a vacuum of approximately 80 mTorr, and the temperature allowed to rise to room temperature, thus inducing sublimation of the ice from the slurry, which is allowed to proceed over 48 hours. The x-ray diffraction trace of the collagen/GAG/brushite triple co-precipitate following removal of unbound water (Cu-K(alpha) radiation) is shown in FIG. 13.

### Step III

After complete removal of unbound water, 1.25 g of the resulting dry triple coprecipitate is hydrated in 40 mL deionised water for 20 minutes.

20 mL of a solution of 0.018M EDAC and 0.007M NHS is added to the container containing the triple coprecipitates and deionised water, and the triple coprecipitates allowed to crosslink for 2 hours at room temperature, under gentle agitation.

The EDAC solution is removed, and the triple coprecipitates are rinsed with phosphate buffer solution (PBS) and allowed to incubate at 37° C. for 2 hours in fresh PBS under mild agitation.

After two hours in PBS, the triple coprecipitates are rinsed with deionised water, and allowed to incubate for two 10-minute intervals at 37° C. under mild agitation.

The triple coprecipitates are then dried at 37° C. for 72 hours. The x-ray diffraction pattern of collagen/GAG/brushite triple coprecipitate following EDAC crosslinking (Cu-K(alpha) radiation) is shown in FIG. 14.

### Step IV

Crosslinked triple coprecipitate granules are placed in 50 mL deionised water pre-saturated with respect to brushite at 37° C., and the pH of the solution adjusted to 8.50 using ammonia.

The temperature and pH are maintained constant for 72 hours, after which time the co-precipitates are filtered, rinsed in deionised water, and dried at 37° C. in air. An x-ray diffraction pattern of the co-precipitates following conversion to apatite is shown in FIG. 15 (EDAC-crosslinked collagen/GAG/CaP triple co-precipitate following conversion at 37° C. to apatite over 72 hours at pH 8.50, to form a collagen/GAG/apatite biocomposite (Cu-K(alpha) radiation).

### Step V

The dried collagen/GAG/Ap triple coprecipitates are subjected to a 32.1 kGy dose of gamma irradiation. FIG. 16 shows the x-ray diffraction pattern following gamma irradiation (EDAC-crosslinked collagen/GAG/Ap triple co-precipitates after secondary crosslinking via gamma irradiation).

### Example 4

### Materials

Collagen: reconstituted, pepsin-extracted porcine dermal collagen (atelocollagen); 85% by weight of Type I, 15% by weight of Type III; Japan Meat Packers (Osaka, Japan)
GAG: Chondroitin-6-sulphate from shark cartilage; sodium salt; Sigma-Aldrich Inc (St. Louis, Mo., USA)
Calcium Sources: (i) Calcium hydroxide; Ca(OH)₂ Sigma-Aldrich Inc (St. Louis, Mo., USA), and (ii) Calcium nitrate; Ca(NO₃)₂.4H₂O; Sigma-Aldrich Inc (St. Louis, Mo., USA)
Phosphorous Source: Orthophosphoric acid; H₃PO₄; BDH Laboratory Supplies (Poole, United Kingdom)

### Titrant: Ammonia; NH₃; BDH Laboratory Supplies (Poole, United Kingdom)

### Procedure

### Step I

Solution A was prepared by dissolving Ca(OH)₂ in 0.48M H₃PO₄ to a concentration of 0.12M at room temperature, and the resulting solution titrated to pH of 3.2.

Suspension B was prepared by dissolving Chondroitin-6-sulphate in deionised water to a concentration of 3.2 g/L. Under constant stirring, Ca(NO₃)₂.4H₂O and Ca(OH)₂ then added to chondroitin sulphate solution at a nitrate:hydroxide molar ratio of 1.5, to produce a suspension with a total calcium concentration of 2.4M.

0.144 g collagen were added to 20 mL of Solution A, and blended using a homogeniser until dissolved. 4 mL of Suspension B was then added to Solution A under constant stirring. Stirring was continued for 60 minutes, and pH monitored to ensure that it remained in the range 3.15<pH<3.30. The resulting slurry was then allowed to age for 24 hours at room temperature.

### Step II

The slurry was allowed to dry at 37° C. in air for 5 days, and the remaining triple co-precipitate rinsed with deionised water, and subsequently dried again at 37° C. for an additional 24 hours.

### Step III

Co-precipitates were placed in dilute acetic acid (pH=3.2), and irradiated with a gamma irradiation dose of 30 kGy. The crosslinked precipitates were then removed from solution, rinsed, and dried at 37° C. in air.

### Step IV

Crosslinked, co-precipitate granules were placed in 50 mL deionised water at 37° C., and the pH of the solution adjusted to 6.65 using ammonia. Temperature and pH were maintained constant for 48 hours, after which the co-precipitates were filtered, rinsed in deionised water, and dried at 37° C. in air.

### Step V

Crosslinked, hydrolysed, co-precipitate granules were placed in a vacuum oven at room temperature, and a vacuum of 50 mTorr applied, after which the temperature was then increased to 105° C. After 24 hours, the temperature was reduced to room temperature and the vacuum released.

FIG. 17 shows the x-ray diffraction pattern of the composite immediately following triple co-precipitation and drying (Steps I and II). This pattern confirms the major phase present to be brushite.

FIG. 18 shows an SEM micrograph of the structure of co-precipitate granules following primary crosslinking (Step III). It is worthy to note the microstructurally homogeneous nature of the granules.

The progression of hydrolysis to octacalcium phosphate (Step IV) is illustrated in the XRD Pattern of FIG. 19. Progressive decreases in the intensity of the brushite peak at 12.5°, and increases of the major octacalcium phosphate(OCP) peak at 4.5° indicate the conversion of the inorganic phase to OCP over a period of 48 hours.

A TEM image of the composite is shown in FIG. 20. A random distribution of 10-20 nm low aspect-ratio calcium phosphate crystals dispersed in a collagen/GAG matrix is evident.

The composite biomaterials of the present invention may be used as a bioresorbable material. Following implantation, it is expected that a device fabricated from the material would resorb completely, leaving behind only healthy, regenerated tissue, with no remaining trace of the implant itself. [End of Annex 1].

1-55. (canceled)

56. A process for the preparation of a composite biomaterial comprising an inorganic material and an organic material, the process comprising:
(a) providing a first slurry composition comprising a liquid carrier, an inorganic material and an organic material;
(b) providing a mould for the slurry;
(c) depositing the slurry in the mould;
(d) cooling the slurry deposited in the mould to a temperature at which the liquid carrier transforms into a plurality of solid crystals or particles;
(e) removing at least some of the plurality of solid crystals or particles by sublimation and/or evaporation to leave a porous composite material comprising an inorganic material and an organic material; and
(f) removing the material from the mould.

57. A process as claimed in claim 56, wherein the inorganic material comprises a calcium phosphate material.

58. A process as claimed in claim 57, wherein the calcium phosphate material comprises brushite.

59. A process as claimed in claim 56, wherein the organic material comprises one or more of collagen (including recombinant human (rh) collagen), a glycosaminoglycan, albumin, hyaluronan, chitosan, and synthetic polypeptides comprising a portion of the polypeptide sequence of collagen.

60. A process as claimed in claim 56, wherein the inorganic material comprises a calcium phosphate material, the organic material comprises collagen and optionally a glycosaminoglycan, and wherein the porous composite material comprises the calcium phosphate material and collagen and optionally a glycosaminoglycan.

61. A process as claimed in claim 60, wherein the first slurry comprises a co-precipitate of collagen and the calcium phosphate material.

62. A process as claimed in claim 60, wherein the first slurry comprises a triple co-precipitate of collagen, the calcium phosphate material and a glycosaminoglycan.

63. A process as claimed in claim 60, wherein the calcium phosphate material comprises brushite.

64. A process as claimed in claim 56 further comprising:
providing a second slurry composition comprising a liquid carrier and an organic material and optionally an inorganic material; and
prior to said cooling step, depositing said second slurry composition in the mould either before or after said first slurry composition has been deposited.

65. A process as claimed in claim 64, wherein the organic material comprises one or more of collagen (including recombinant human (rh) collagen), a glycosaminoglycan, albumin, hyaluronan, chitosan, and synthetic polypeptides comprising a portion of the polypeptide sequence of collagen.

66. A process as claimed in claim 64, wherein the second slurry composition comprises an inorganic material, preferably a calcium phosphate material.

67. A process as claimed in claim 64, wherein the second slurry composition comprises a liquid carrier, collagen, optionally a calcium phosphate material, and optionally a glycosaminoglycan.

68. A process as claimed in claim 67, wherein the second slurry composition comprises a co-precipitate of collagen and a glycosaminoglycan, or a co-precipitate of collagen and a calcium phosphate material, or a triple co-precipitate of collagen, a glycosaminoglycan and a calcium phosphate material.

69. A process as claimed in claim 67, wherein the second slurry composition comprises a calcium phosphate material which is brushite.

70. A synthetic composite biomaterial comprising:
a first layer formed of a porous material comprising collagen and a calcium phosphate material and optionally a glycosaminoglycan; and
a second layer joined to the first layer and formed of a material comprising collagen, or a co-precipitate of collagen and a glycosaminoglycan, or a co-precipitate of collagen and a calcium phosphate material, or a triple co-precipitate of collagen, a glycosaminoglycan and a calcium phosphate material.

71. A biomaterial as claimed in claim 70, wherein the first layer is formed of a biomaterial wherein at least part of the biomaterial is formed from a porous co-precipitate comprising a calcium phosphate material and one of collagen (including recombinant human (rh) collagen), a glycosaminoglycan, albumin, hyaluronan, chitosan or a synthetic polypeptides comprising a portion of the polypeptide sequence of collagen, wherein the macropore size range (pore diameter) is preferably from 1-1000 microns, more preferably from 200-600 microns.

72. A biomaterial as claimed in claim 70, wherein the first and second layers are integrally formed, preferably by liquid phase co-synthesis.

73. A biomaterial as claimed in claim 71, wherein the first and second layers are joined to one another through an inter-diffusion layer.

74. A biomaterial as claimed in claim 71, wherein the first and second layers are joined to one another through an inter layer.

75. A biomaterial as claimed in claim 71, wherein the second layer is porous or non-porous.

76. A biomaterial as claimed in claim 71, comprising one or more further layers joined to the first and/or second layers, each of said further layers being formed of a material comprising collagen, or a co-precipitate of collagen and a glycosaminoglycan, or a co-precipitate of collagen and a calcium phosphate material, or a triple co-precipitate of collagen, a glycosaminoglycan, and at least one calcium phosphate material.

77. A biomaterial as claimed in claim 76, wherein the first and second layers and said one or more further layers are integrally formed.

78. A biomaterial as claimed in claim 76, wherein adjacent layers are joined to one another through an inter-diffusion layer.

79. A biomaterial as claimed in claim 76, wherein at least one of said one or more further layers is/are porous or non-porous.

80. A biomaterial as claimed in claim 71, wherein the material comprises collagen and a glycosaminoglycan, and wherein the collagen and glycosaminoglycan are crosslinked.

81. A synthetic bone material, bone implant, bone graft, bone substitute, bone scaffold, filler, coating or cement comprising a biomaterial as defined in claim 70.

82. A monolithic or layered bone scaffold for use in tissue engineering comprising a biomaterial as defined in claim 70.
